SEQUENCE LISTING (5 sequences)

SEQ ID NO: 1 - DNA, 12 bp, synthetic construct
  source 1..12: mol_type = other DNA
ttgcgtccagaa

SEQ ID NO: 2 - DNA, 12 bp, synthetic construct
  source 1..12: mol_type = other DNA
ttacgacctgaa

SEQ ID NO: 3 - DNA, 12 bp, synthetic construct
  source 1..12: mol_type = other DNA
ttgaggcctgag

SEQ ID NO: 4 - DNA, 12 bp, synthetic construct
  source 1..12: mol_type = other DNA
tttcgacctaaa

SEQ ID NO: 5 - DNA, 12 bp, synthetic construct
  source 1..12: mol_type = other DNA
ttgtatcctgag

(19) United States
(12) Patent Application Publication
(10) Pub. No.: US 2024/0321356 A1
(43) Pub. Date: Sep. 26, 2024

(54) EFFICIENT INFORMATION CODING IN LIVING ORGANISMS

(71) Applicant: RAMOT AT TEL-AVIV UNIVERSITY LTD., Tel Aviv (IL)

(72) Inventors: Tamir TULLER, Tel Aviv (IL); Itzhak TAMO, Tel Aviv (IL); Alon AKIVA, Tel Aviv (IL)

(21) Appl. No.: 18/733,444

(22) Filed: Jun. 4, 2024

Related U.S. Application Data
(63) Continuation of application No. PCT/IL2022/051291, filed on Dec. 5, 2022.
(60) Provisional application No. 63/286,073, filed on Dec. 5, 2021.

(51) Int. Cl. G11C 13/02; G16B 30/20

(52) U.S. Cl. CPC G11C 13/02; G16B 30/20

(57) ABSTRACT

Methods of storing information in a living organism are provided. Systems and computer program products for performing the methods are also provided.

### CROSS REFERENCE TO RELATED APPLICATIONS

This application is a bypass continuation of PCT/IL2022/051291 filed Dec. 5, 2022, which claims the benefit of priority of U.S. Provisional Patent Application No. 63/286,073, Dec. 5, 2021 entitled “EFFICIENT INFORMATION CODING IN LIVING ORGANISMS.” The contents of both applications are incorporated herein by reference in their entireties.

### REFERENCE TO AN ELECTRONIC SEQUENCE LISTING

The contents of the electronic sequence listing (RMT-P-015-PCT.xml; Size: 5,206 bytes; and Date of Creation: Dec. 4, 2022) is herein incorporated by reference in its entirety.

### FIELD OF INVENTION

The present invention is in the field of information coding in living organisms.

### BACKGROUND OF THE INVENTION

It is clear that DNA can be used as a storage medium, each nucleotide carrying two information bits, which can store vast amounts of data for very long periods of time and with high reliability, as over time humans remains human just as cats remain cats. However, it is also clear that it is a very complex storage medium affected by evolutionary processes which create various data corruption mechanisms as well as complex constraints over the stored information, where even a slight change in the nucleotides sequence in the DNA may profoundly affect the whole organism's behavior and possibly decrease its growth rate, increase the mutation rate and may even cause the death of the organism. To store information successfully over a living organism, all of the above processes must be considered. A new model to store information in this way is greatly needed.

### SUMMARY OF THE INVENTION

The present invention provides methods of storing information in a living organism. Systems and computer program products for performing the methods of the invention are also provided.

According to a first aspect, there is provided a method of storing information in a living organism, the method comprising:
a. receiving a genomic sequence of the living organism;
b. selecting a fragment of a coding region within the received genomic sequence in which to store the information, wherein the selecting comprises:
   i. estimating indel probability across coding regions of the genomic sequence and selecting fragments of the coding regions with a probability below a predetermined threshold for all nucleotides within the fragment;
   ii. receiving coding regions of orthologs of the organism and selecting fragments of the coding regions of the living organism with an intermediate nucleotide sequence identity probability across the orthologs; and
   iii. selecting a fragment of a coding region that was selected in both (i) and (ii).; and
c. inducing genetic mutations in the selected fragment of a coding region in a cell of the living organism such that the genetic mutations are convertible to the information, wherein the mutations are not substantially detrimental to the health of the living organism;
thereby storing information in a living organism.

According to some embodiments, the fragment is between 150-350 codons in length.

According to some embodiments, the fragment is between 180 and 200 codons in length.

According to some embodiments, the living organism is a single celled organism.

According to some embodiments, the living organism is a bacterium.

According to some embodiments, the selected fragment comprises an indel probability variance below a predetermined threshold for all nucleotides within the fragment.

According to some embodiments, the indel probability variance threshold is 0.05.

According to some embodiments, coding regions from at least 100 orthologs are received.

According to some embodiments, an intermediate identity probability is between 0.55 and 0.75.

According to some embodiments, the not substantially detrimental mutation is a synonymous mutation.

According to some embodiments, the not substantially detrimental mutation is a mutation to a codon that appears within at least a predetermined threshold percentage of orthologs.

According to some embodiments, the predetermined threshold percentage is 5%.

According to some embodiments, the fragment is devoid of at least 2 consecutive nucleotides which cannot be mutated without being substantially detrimental to the health of the living organism.

According to some embodiments, the inducing genetic mutations comprises optimizing for the living organism the codon adaptation index (CAI) within the fragment.

According to some embodiments, an optimized CAI is a CAI with the smallest change from the unmutated fragment sequence.

According to some embodiments, the genetic mutations also include information of a numeric identifier which identifies the order in which the information was stored in a plurality of fragments.

According to some embodiments, a first or second codon of the fragment is mutated to include information of the numeric identifier.

According to some embodiments, the stored information retains at least 90% fidelity after at least 50 generations of the genetically mutated living organism.

According to some embodiments, information is encoded into the mutations using an error correcting code.

According to some embodiments, the error correcting code is a Reed-Solomon (RS) code.

According to some embodiments, the information is encoded using a modulation algorithm.

According to some embodiments, the fragment comprises mutations convertible to a numeric identifier and the mutations convertible to the information and the mutations convertible to the numeric identifier are encoded separately.

According to some embodiments, the method further comprises reading the stored information, wherein the reading comprises:
d. receiving by a reader, sequences of coding regions of the genetically mutated living organism or a descendant of the genetically mutated living organism;
e. identifying the fragments comprising the stored information;
f. correcting any indels and any point mutations within the fragment that arose since the inducing; and
g. extracting the information from the induced genetic mutations.

According to some embodiments, the identifying fragments comprises comparison to the native genome of the living organism.

According to some embodiments, the fragment locations and known and the identifying comprises locating the fragments within the received sequences.

According to some embodiments, the method further comprises between step (e) and (f), a step comprising reading a numeric identifier in a plurality of fragments and ordering the fragments based on the read numeric identifiers.

According to some embodiments, the method further comprises removing the numeric identifiers from the fragments and concatenating the fragments in an order established by the read numeric identifiers.

According to some embodiments, the reading a numeric identifier in a plurality of fragments comprises decoding the numeric identifiers separately from the stored information.

According to some embodiments, the reading comprises maximum likelihood decoding which ensures all ordinal numbers up to the total number of identifiers are represented in the numeric identifiers by selecting the numeric identifier with the highest probability of being each ordinal.

According to some embodiments, the correcting comprises employing an error correcting code decoder.

According to some embodiments, the error correcting code decoder is a RS decoder.

According to some embodiments, the method comprises storing information in a plurality of different organisms and wherein the numeric identifier identifies the order of the information across the plurality of different organisms.

According to another aspect, there is provided a system comprising:
at least one hardware processor; and
a non-transitory computer-readable storage medium having stored thereon program code, the program code executable by the at least one hardware processor to perform a method of the invention.

According to another aspect, there is provided a computer program product comprising a non-transitory computer-readable storage medium having program code embodied therewith, the program code executable by at least one hardware processor to perform a method of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Diagram of the Noisy Shuffling Sampling Channel.

FIG. 2: Diagram of the DNA writing process. Information bits are stored over multiple DNA sequences together by changing their original content. Specific nucleotides will be changed, depicted in red letters, only in a fixed area in the DNA which we call a site and is shown in green.

FIG. 3: Diagram of the Living Organism Channel model. DNA sequences may erase, permute, and specific nucleotides may substitute.

FIG. 4: Diagram of the Living Organism Channel, represented as a concatenation of the Random Subsets Channel and the Noisy Shuffling Sampling Channel.

FIG. 5: Diagram of the DNA reading process. Multiple DNA sequences are read, and the information stored over them is reconstructed. The read DNA sequences are corrupted by the Living Organism Channel, which may erase sequences, permute sequences and substitute nucleotides (as shown in red letters).

FIG. 6: Diagram of a system model of DNA storage over living organisms. An information message is written by changing the DNA contents of multiple DNA sequences of multiple microorganisms. The information is corrupted over time by the Living Organism Channel. Finally, the DNA sequences are read from a pool of unordered microorganisms, and the information message is reconstructed.

FIG. 7: Chart showing the Noisy Shuffling Sampling Channel Capacity Bounds, as a function of the erasure and substitution probabilities, for 180 DNA sequences of 144 bits each. The classic capacity term (in the asymptotic regime) gives the upper bound and the lower bound by the derivation of this section.

FIG. 8: Chart showing the Noisy Shuffling Sampling Channel Capacity Bounds Degradation, showing the normalized ratio between the difference of the higher and lower bound, relative to the higher bound.

FIG. 9: Charts showing the Noisy Shuffling Sampling Channel Capacity Bounds Degradation, as a function of the nucleotides substitution probability (top) and the DNA sequence erasure probability (bottom).

FIG. 10: Diagram of the Random Subsets Channel setting, defined over a discrete and finite alphabet and with an i.i.d state vector.

FIG. 11: Diagram of the Random Subsets Channel with side information at the transmitter is the relevant scenario to our DNA storage problem.

FIG. 12: Line graph of the lower bound for the Completely Random Subset Channel Capacity. The lower bound is given in bits per channel use and as a function of the alphabet size. Each plotted graph is for a different subset size.

FIG. 13: Graphs showing the Modulo Random Subsets Code Rate. The rate is given in bits per channel use and as a function of the subset size. The alphabet size is assumed to be large compared to the subset size. The lower graph shows the coding rate gap, defined as the ratio between the construction's code rate to the achievable rate in Theorem 6.

FIG. 14: Graphs showing the Monte Carlo evaluation of the code rate of the Linear Random Subsets Code Construction, compared to the Completely Random Subsets achievable rate (Theorem 6) and the Modulo Random Subsets Code Construction, for an alphabet size much bigger than the subsets size.

FIGS. 15A-C: (15A) Graphs of the Gene ALA1 identity (top) and indel estimations (bottom) after DNA alignment. The identity probability is calculated as the relative number of appearances of the most likely codon among all Orthologs in each location. The indel probability is calculated as the relative number of gap insertions among all Orthologs in each location. (15B) Graph of the ALA1 indel estimation along the site of interest. There is a high estimation correlation along all nucleotides. (15C) Graph of ALA1 identity probability along the site of interest.

FIG. 16: Graph of dictionary mapping sets size for a site of interest. The subsets sizes were calculated as the number of codons that follows one out of 3 different criteria in each DNA location and among all the Orthologs in which the DNA alignment was performed.

FIG. 17: Diagram of the Encoder. The whole information message is encoded using Reed-Solomon code and then divided into message blocks. An identifier is attached to each message block.

FIG. 18: Diagram of the Modulator. Each message block is translated into nucleotides, and the Linear Random Subsets encoder is performed over each message block.

FIG. 19: Modulator scheme for a single synthesized site. Each subsite must obey the dictionary constraint, an algebraic equation, and finally, the CAI optimal option is chosen.

FIG. 20: Diagram of the Demodulator. Each read site is demodulated using the Linear Random Subsets decoder and retranslated into a binary vector.

FIG. 21: Diagram of the Decoder. All the binary vectors are sorted using the identifiers and then decoded using a Reed-Solomon decoder.

FIG. 22: Graph of Encoder-Decoder simulation result, compared to theoretical bounds. Only the encoder, Noisy Shuffling Sampling Channel and decoder were simulated.

FIG. 23: Graph of Encoder-Decoder rate degradation compared to the achievable lower bound.

FIG. 24: Graph of Encoder-Decoder rate degradation compared to the achievable lower bound as a function of the substitution probability (first) and the erasure probability (second).

FIG. 25: Graph of Encoder-Decoder coding rates comparison. The green edged graph shows the performance of our coding scheme, and the blue edged graph the performance of the proposed scheme in Shomorony and Heckel.

FIG. 26: Graph of failure probability of both Encoder-Decoder schemas when fixing the first schema's failure rate.

FIG. 27: Graph of failure probability of both Encoder-Decoder scheme as when fixing the second schema's failure rate.

FIG. 28: Graph of coding rates both Encoder-Decoder schemas, when fixing both failure probabilities.

FIG. 29: Flow chart of an embodiment of a method of the invention.

FIG. 30: Graph of the Noisy Shuffling Sampling Capacity bounds, M=64, L=144. The higher bound is given by the classic capacity and the lower bound by the derivation provided herein.

FIG. 31: Diagram of the Modulator scheme for a single synthesized site. Each subsite must obey the dictionary constraint, an algebraic equation, and finally, the CAI optimal option is chosen.

### DETAILED DESCRIPTION OF THE INVENTION

The present invention, in some embodiments, provides methods of storing information in a living organism. Systems and computer program products for performing the methods are also provided.

DNA synthesis in living cells imposes different mechanisms of data corruption and requires new sophisticated analysis methods as compared to artificial gene synthesis. The invention is based on surprising findings when performing information coding over living organisms from a digital communication system perspective. Living organism were modeled as a discrete communication channel that captures the significant biological phenomena and derives the channel capacity for the model as a function of those major biological parameters. Then, computational molecular evolution approaches were used to estimate those important biological parameters. Next, a coding scheme that allows reliable communication over this channel was enacted and its performance was analyzed. The design goals of the coding scheme are efficiency (in terms of storage density), flexibility (in terms of scalability and minimization of environment dependency), and decoder simplicity (in terms of minimal side information needed). Numerical simulations were performed over actual genomic data based on microorganism models to evaluate the approach. The simulations include random channel behavior based on parameter estimations from evolutional models and genomic data. In addition, an experimental framework for evaluating the model was designed and the final method was found to have high fidelity over hundreds of generations of the organism.

The modern era created demands for data storage for many different applications and reasons. Therefore, the search for efficient storage media is perpetual, and DNA was not skipped over. Much theoretical research exploring the mathematical framework and analysis of artificial DNA synthesis has been done. In Shomorany and Heckel, “DNA-based storage: models and fundamental limits”, arxiv.org/abs/2001.06311, herein incorporated by reference in its entirety, the authors introduced a new communication channel called the Noisy Shuffling Sampling Channel, dealing with artificial DNA strands that suffer from erasures, substitutions, and permutations. Other works that discuss in detail coding over multiple synthesized DNA strands, show that they suffer from substitutions of nucleotides and random permutation of DNA strands. It has been shown that simple indexing is not optimal to reorder permuted DNA strands, however this was demonstrated in a channel model that does not include DNA strand erasures.

By a first aspect, there is provided a method of storing information in a living cell, the method comprising:
a. receiving a genomic sequence of the cell;
b. selecting a fragment of a coding region within the received genomic sequence in which to store the information; and
c. inducing genetic mutation in the selected fragment of a coding region in a living cell such that the genetic mutations are convertible to the information;
   thereby storing information in a living cell.

In some embodiments, the method is an in vitro method. In some embodiments, the method is an in vivo method. In some embodiments, the cell is alive. In some embodiments, the cell is a prokaryotic cell. In some embodiments, the cell is a eukaryotic cell. In some embodiments, the cell is an organism. In some embodiments, the organism is a single cell organism. In some embodiments, the organism is a microorganism. In some embodiments, the cell is a bacterial cell. In some embodiments, the organism is a bacterium. In some embodiments, the cell is a part of an organism. In some embodiments, the cell is in culture. In some embodiments, the cell is in a laboratory. In some embodiments, the cell is in the wild.

In some embodiments, a genomic sequence is a whole genome. In some embodiments, a genomic sequence is a portion of a genome. In some embodiments, the portion is at least 10, 15, 20, 25, 30, 35, 40, 45, 50, 55, 60, 65, 70, 75, 80, 85, 90, 95, 97, 99 or 100%. Each possibility represents a separate embodiment of the invention. In some embodiments, the genomic sequence is a coding region. In some embodiments, the genomic sequence comprises a coding region. In some embodiments, the portion is all coding regions. In some embodiments, the portion is a portion of coding regions. In some embodiments, the genomic sequence is a gene. In some embodiments, the genomic sequence is all genes. In some embodiments, the genomic sequence is a portion of all genes. In some embodiments, the genomic sequence is of the cell. In some embodiments, the genomic sequence is of the organism.

In some embodiments, a fragment is at least 4, 5, 10, 20, 25, 30, 40, 50, 60, 70, 80, 90, 100, 120, 130, 140, 150, 160, 170, 180, 190, or 200 codons. Each possibility represents a separate embodiment of the invention. In some embodiments, a fragment is at least 4 codons. In some embodiments, a fragment is at least 100 codons. In some embodiments, a fragment is at least 180 codons. In some embodiments, a fragment is at least 200 codons. In some embodiments, a fragment is at most 180, 190, 200, 210, 220, 230, 240, 250, 260, 270, 280, 290, 300, 350, 400, 450, 500, 600, 700, 800, 900 or 1000 codons. Each possibility represents a separate embodiment of the invention. In some embodiments, a fragment is between 150-350 codons. In some embodiments, a fragment is between 180-350 codons. In some embodiments, a fragment is between 200-350 codons. In some embodiments, a fragment is between 150-200 codons. In some embodiments, a fragment is between 150-180 codons. In some embodiments, a fragment is between 180-200 codons.

In some embodiments, selecting comprises estimating indel probability across coding regions of the genomic sequence. As used herein the term “indel” refers to an insertion of deletion of nucleic acid bases. In some embodiments, coding regions is at least 2, 3, 4, 5, 6, 7, 8, 9, 10, 20, 30, 40, 50, 60, 70, 80, 90, 100, 200, 300, 400, 500, 600, 700, 800, 900 or 1000 coding regions. Each possibility represents a separate embodiment of the invention. In some embodiments, coding regions is all coding region in the genomic sequence. In some embodiments, coding region is all coding regions in the genome of the cell. In some embodiments, selecting comprises selecting a fragment of the coding regions with an indel probability below a predetermined threshold. In some embodiments, all fragments with an indel probability below the threshold are selected. In some embodiments, the probability if for the whole fragment. In some embodiments, the probability is for all nucleotides with the fragment. In some embodiments, the probability is calculated for the whole fragment. In some embodiments, the method further comprises estimating or calculating the indel probability. In some embodiments, the indel probability is calculated using equation (27). In some embodiments, the indel probability is calculated for all fragments with an intermediate identity probability. In some embodiments, the average indel probability for the whole fragment is below the threshold. In some embodiments, all nucleotides of the fragment are below the indel threshold. In some embodiments, the indel probability variance across all the nucleotides within the fragment is below a predetermined threshold.

In some embodiments, the indel probability threshold is 0.00001, 0.00005, 0.0001, 0.0005, 0.001, 0.005, 0.01, 0.02, 0.03, 0.04, 0.05, 0.06, 0.07, 0.08, 0.09, or 0.1. Each possibility represents a separate embodiment of the invention. In some embodiments, the probability is a percent probability. In some embodiments, the indel probability threshold is 0.05. In some embodiments, the indel variance threshold is 0.05. In some embodiments, the indel probability threshold is 5%.

In some embodiments, selecting further comprises receiving coding regions of orthologs of the cell. In some embodiments, selecting further comprises receiving orthologous coding regions to the coding regions of the cell. In some embodiments, selecting further comprises receiving orthologous coding regions to the received coding regions of the cell. In some embodiments, selecting further comprises receiving coding regions of orthologs of the organism. In some embodiments, the coding regions of the ortholog correspond to the coding regions of the cell. In some embodiments, the orthogonal coding regions are received. It will be understood by a skilled artisan that if only a portion of coding regions of the cell are received then the corresponding/orthologous coding regions from the orthologs must be received. In some embodiments, an ortholog is a homolog. As used herein, “orthologs” and “orthologous” refer to the same genes in different species. In some embodiments, the different species are species that have evolved through speciation events only. In some embodiments, orthologs evolved from the same ancestral gene. In some embodiments, orthologs comprise the same function. In some embodiments, orthologs comprise at least 30, 40, 50, 55, 60, 65, 70, 75, 80, 85, 90, 92, 95, 97 or 99% identity. Each possibility represents a separate embodiment of the invention. In some embodiments, identity is on the amino acid level. In some embodiments, identity is on the nucleotide level. In some embodiments, orthologs comprise at least 30, 40, 50, 55, 60, 65, 70, 75, 80, 85, 90, 92, 95, 97 or 99% homology. Each possibility represents a separate embodiment of the invention. In some embodiments, homology is on the amino acid level. In some embodiments, homology is on the nucleotide level. In some embodiments, at least 10, 20, 30, 40, 50, 60, 70, 80, 90, 100, 125, 150, 175, 200, 250, 300, 350, 400, 450, or 500 orthologs are received. Each possibility represents a separate embodiment of the invention. In some embodiments, at least 100 orthologs are received.

In some embodiments, the region comprises an average of allowed changes of at least a predetermined threshold number. In some embodiments, allowed changes are allowed codon changes. In some embodiments, the average is the average number of allowable substitute codons to which a given codon maybe mutated. In some embodiments, thet average is the average for a codon of the region. In some embodiments, the average is the average of the region. In some embodiments, the predetermined threshold is at least 4 codons. In some embodiments, the predetermined threshold is at least about 4.5 codons. In some embodiments, the region is devoid of at least 2 consecutive nucleotides which cannot be mutated. It will be understood that mutations can be made to a nucleotide only if they will result in an allowable codon change. Some nucleotides will be invariable, in that they cannot be mutated and not violate the rules for allowable codons.

In some embodiments, selecting fragments comprises calculating for fragments from the cell a nucleotide sequence identity probability across the orthologs. In some embodiments, an identity probability is an identity probability of all nucleotides in a site. In some embodiments, an identity probability is the probability of substitution over a predetermined number of generations. In some embodiments, the predetermined number of generations is at least 10, 15, 20, 25, 30, 35, 40, 45, 50, 60, 70, 75, 80, 90 or 100. Each possibility represents a separate embodiment of the invention. In some embodiments, selecting fragments comprises calculating for fragments from the cell a nucleotide sequence identity probability across the orthologous sequences to a given fragment. It will be understood by a skilled artisan that for a given fragment sequence, each nucleotide base can be compared to all corresponding nucleotide bases in all the orthologs and the percentage of orthologs that have that exact base (the identity score) can be calculated. For example, for a given T nucleotide, 100 orthologous sequences are examined. A T base is found at this exact position in 75 of the orthologs and so for this base the identity probability is 75%. The average identity probability for the fragment is the average of the scores for each base. In some embodiments, the identity is calculated for all fragments. In some embodiments, the identity is calculated for all fragments with an indel probability below the threshold.

In some embodiments, selecting fragments comprises selecting fragments with a nucleotide identity probability above a predetermined threshold. In some embodiments, selecting fragments comprises selecting fragments with a nucleotide identity probability below a predetermined threshold. In some embodiments, selecting fragments comprises selecting fragments with an intermediate nucleotide identity probability. In some embodiments, intermediate is above a first predetermined threshold and below a second predetermined threshold. In some embodiments, the first threshold is the lower threshold. In some embodiments, the second threshold is the upper threshold. The lower threshold is the level below which we are concerned about random mutation due to a lack of evolutionary pressure on the sequence. The upper threshold is the level above which we are concerned that an induced genetic mutation would damage the function of the protein and/or the viability of the cell. An intermediate identity probability meets all criteria as the region is sufficiently conserved that random mutations are unlikely, but not so conserved that the method does not have mutations that can be made without damaging the cell's health.

In some embodiments, the lower threshold is 0.2, 0.25, 0.3, 0.35, 0.4, 0.45, 0.5, 0.55, 0.6, 0.65 or 0.7. Each possibility represents a separate embodiment of the invention. In some embodiments, the lower threshold is 0.55. In some embodiments, threshold is a percent. In some embodiments, the lower threshold is 55%. In some embodiments, the lower threshold is 0.7. In some embodiments, the upper threshold is 0.7, 0.75, 0.8, 0.85, 0.9, 0.95, 0.97 or 0.99. Each possibility represents a separate embodiment of the invention. In some embodiments, the upper threshold is 0.75. In some embodiments, threshold is a percent. In some embodiments, the lower threshold is 75%. In some embodiments, the upper threshold is 0.7. In some embodiments, intermediate is between 0.55-0.75. In some embodiments, intermediate is about 0.7.

In some embodiments, selecting fragments comprises selecting fragments that meet both the indel and identity criteria. In some embodiments, at least one fragment is selected. In some embodiments, a plurality of fragments is selected. In some embodiments, a sufficient number of fragments are selected to encode the information.

In some embodiments, the information is a message. In some embodiments, the information is test. In some embodiments, the information is encoded. In some embodiments, the information is binary information. In some embodiments, the information is transformed into bits. In some embodiments, code is used to convert the information into bits. In some embodiments, the code is ASCII encoding. In some embodiments, 1 character of the information is converted into 1 bit. In some embodiments, the information is encoding into bits. In some embodiments, two bits are encoded into a single nucleotide. For example, 00=A, 01=T, 10=C and 11=G. In some embodiments, the encoding is with an error correcting code. In some embodiments, the error correcting code is Reed-Solomon code. In some embodiments, the Reed-Solomon code is a shortened Reed-Solomon code.

In some embodiments, the mutations are not detrimental to the health of the cell. In some embodiments, detrimental is substantially detrimental. In some embodiments, detrimental is measurably detrimental. In some embodiments, the health of the cell is survival of the cell. In some embodiments, health of the cell is dividing time of the cell. In some embodiments, health of the cell is doubling time of the cell. In some embodiments, health of the cell is robustness of the cell. In some embodiments, health of the cell is the ability of the cell to compete with other cells in the environment. Cell heath, doubling time and survival can be measured by any method known in the art. These include measuring OD of the cells in culture, counting cells, and staining cells with a viability dye to name but a few.

In some embodiments, a nondetrimental mutation is a mutation to a synonymous codon. In some embodiments, a nondetrimental mutation is a mutation that is found in a dictionary of allowable mutations. In some embodiments, the dictionary is all synonymous mutations. In some embodiments, a nondetrimental mutation is a mutation to a codon at appears in at least a predetermined threshold percentage of orthologs. In some embodiments, the dictionary is the mutations found in orthologs at least a predetermined threshold frequency. In some embodiments, the ortholog threshold is 0.00001, 0.00005, 0.0001, 0.0005, 0.001, 0.005, 0.01, 0.02, 0.03, 0.04, 0.05, 0.06, 0.07, 0.08, 0.09, or 0.1. Each possibility represents a separate embodiment of the invention. In some embodiments, the probability is a percent probability. In some embodiments, the ortholog threshold is 0.05. In some embodiments, the ortholog threshold is 5%. Thus, for example, is a mutation is found in at least 5% of orthologs it is considered a nondetrimental mutation and so may be made as part of the encoding of the information.

In some embodiments, inducing genetic mutation comprises optimizing for the cell the codon usage bias (CUB). In some embodiments, the CUB is calculated by the codon adaptation index (CAI). In some embodiments, optimizing is with the fragment. In some embodiments, the optimizing if for the genomic region. In some embodiments, the optimizing is for the genome. In some embodiments, the CUB is the cell's CUB. In some embodiments, the CUB is the organism's CUB. In some embodiments, the CAI is the cell's CAI. In some embodiments, the CAI is the organism's CAI. In some embodiments, CUB optimization is by CAI. In some embodiments, an optimized CAI is a CAI with the smallest change from the unmutated fragment sequence. Optimization of CAI when encoding greatly improves the method of the invention as it optimizes organism survival and decreases loss of message information. This is similar to the effect produced by the use of a dictionary of disallowed codon changes. Together, both of these elements help to ensure organism survival (decreases organism loss), decrease message loss/mutation and generally improve the fidelity of the message and allow for longer storage times.

These differences are quantified by various “codon usage bias (CUB) indices” which represent the selective pressure acting on synonymous codons and are therefore correlated with translation efficiency. The codons are scored independently and given 3 different measurements.

CAI: Codon Adaptation Index: This measurement is the most broadly used CUB analysis tool-ideally, a set of highly expressed ORFs is composed and is assumed to have higher selective pressure for translation efficiency due to their expression levels.

For each group of synonymous codons (f), the frequency of each amino acid (fᵢ) is normalized by the highest frequency of the codons in that group, for the entire reference set:

$${w}_{i} = \frac{{f}_{i}}{max ( {f}_{i} )} .$$

In some embodiments, the information is encoded into the mutations. In some embodiments, the mutations encode the information. In some embodiments, the information is encoded with an error correcting code. In some embodiments, the error correcting code is a Reed-Solomon (RS) code. Error correcting codes are well known in the art and any such code may be employed. In some embodiments, the information is encoding using a modulation algorithm. Modulation algorithms are well known in the art and described hereinbelow. Any such algorithm may be employed.

In some embodiments, the genetic mutations also include information of an identifier. In some embodiments, the method further comprises producing genetic mutation convertible to an identifier. In some embodiments, the method further comprises encoding an identifier into the fragment. In some embodiments, the identifier is a numeric identifier. In some embodiments, the identifier is an ordinal identifier. In some embodiments, the identifier identifies the order in which the information was stored in the fragments. In some embodiments, the fragments are a plurality of fragments. In some embodiments, the mutations convertible to the information and the mutations convertible to the identifier are encoded separately. In some embodiments, the information and the identifier are encoded separately. In some embodiments, each identifier is encoded separately. In some embodiments, all identifiers are encoded together. In some embodiments, the identifier is encoded with greater fidelity than the information. In some embodiments, the identifier is encoded with a lower coding rate than the information. In some embodiments, the identifier is encoded with a higher coding rate than the information. It will be understood by a skilled artisan that the identifiers and the message are each separately protected by their own error correcting code. That is an additional RS code is used to encode the identifiers. This has the advantage of allowing the identifiers to be decoded successfully even if the message cannot be (e.g., due to mutation or indels). The use of separate codes greatly improves the method and ensures proper ordering as well as greater messaging accuracy.

In some embodiments, the fragments are from a plurality of organisms. In some embodiments, the organisms are different organisms. It will be understood by a skilled artisan that the identifiers allow the ordering of the fragments by a reader into the order in which they were encoded. In some embodiments, the identifier is encoded in the same manner as the information. In some embodiments, the identifier is encoded by a different code than the information. In some embodiments, the identifiers and information have different coding matrixes. In some embodiments, the identifier is incorporated into the information. In some embodiments, the identifier is encoded with an error correcting code. In some embodiments, a first codon of the fragment is mutated to include information of the identifier. In some embodiments, a second codon of the fragment is mutated to include information of the identifier. In some embodiments, a first or second codon of the fragment is mutated to include information of the identifier. In some embodiments, the information of the identifier is encoded into the 5′ end of the fragment. In some embodiments, the 5′ end is within the first 1, 2, 3, 4, 5, 6, 7, 8, 9 or 10 codons. Each possibility represents a separate embodiment of the invention. In some embodiments, the identifier comprises a coding rate of at most 0.5. It will be understood that such a coding rate is intended to provide maximum protection for the identifier and a lost identifier will result in complete loss of the fragment of the message it was identifying. In some embodiments, the identifier comprises 6 bits. In some embodiments, the coding rate is 2 bits per nucleotide.

In some embodiments, the stored information retains a high fidelity. In some embodiments, high is at least 50, 55, 60, 65, 70, 75, 80, 85, 90, 92, 95, 97, 99 or 100% identity. Each possibility represents a separate embodiment of the invention. n some embodiments, high is at least 90%. In some embodiments, the fidelity is maintained for at least 10, 20, 30, 40, 50, 60, 70, 80, 90, 100, 150, 200, 250, 300, 350, 400, 450, 500, 600, 700, 800, 900 or 1000 generations. Each possibility represents a separate embodiment of the invention. In some embodiments, a generation is a cell division. In some embodiments, a generation is a cell population doubling. In some embodiments, a generation is an offspring of the cell. In some embodiments, a generation is an offspring of the organism. In some embodiments, a generation is a generation of the mutated cell. In some embodiments, a generation is a generation of the mutated organism. In some embodiments, the fidelity is maintained for at least 1, 2, 3, 4, 5, 6, 7, 8, 9, 10, 12, 15, 17, 20, 22, 25, 27, 30, 35, 40, 45 or 50 years. Each possibility represents a separate embodiment of the invention. In some embodiments, fidelity is maintained for at least 1 year. In some embodiments, fidelity is maintained for at least 5 years. In some embodiments, fidelity is maintained for at least 10 years.

In some embodiments, the method further comprises reading the stored information. In some embodiments, the reading is by a different entity that performed the storing. In some embodiments, the reading is by an entity that does not know the code. In some embodiments, the reading is by an entity that knows the code. In some embodiments, the reading is by an entity possessing the passcode. In some embodiments, the reading is by an entity that knows the codeword. In some embodiments, the reading is by an entity that knows the keyword. In some embodiments, the reading is by an entity that does not know the keyword/passcode/codeword. In some embodiments, the reading is by an entity that known the locations of the fragments. In some embodiments, the reading is by an entity that does not know the location of the fragments.

In some embodiments, the reading comprises receiving by a reader sequences of genomic sequences of the genetically mutated cell or a descendant of the genetically mutated cell. In some embodiments, the reading comprises receiving by a reader sequences of coding regions of the genetically mutated cell or a descendant of the genetically mutated cell. In some embodiments, the sequences are reads of whole genome sequencing. In some embodiments, the sequences are a genome of the cell. The reader must receive the stored information in the form of genomic sequences in order to readout the information. In some embodiments, the method comprises identifying the fragments comprising the stored information. In some embodiments, the method comprises correcting any indels within the fragments. In some embodiments, the reading comprises correcting any indels within the fragments. In some embodiments, the method comprises correcting any point mutation within the fragments. In some embodiments, the reading comprises correcting any point mutation within the fragments. In some embodiments, the indels are indels that arose since the inducing. In some embodiments, the indels are indels that arose since the storing. In some embodiments, the mutations are mutations that arose since the inducing. In some embodiments, the mutations are mutations that arose since the storing. In some embodiments, the reading further comprises extracting the information from the induced genetic mutations. In some embodiments, the reading further comprises extracting the information from the fragments. In some embodiments, the extracting is by means of a decoder. In some embodiments, the correcting is by means of a decoder. In some embodiments, the decoder is a decoder of the error correcting code. In some embodiments, the correcting comprises employing a RS decoder. In some embodiments, the reading is without knowing the location of the fragments. In some embodiments, the reading is with knowledge of the location of the fragments. In some embodiments, the decoder has only the coding matrix. In some embodiments, the coding matrix is the inner coding matrix.

In some embodiments, identifying fragments comprises comparison to a native genome of the cell. In some embodiments, identifying fragments comprises comparison to a non-modified genome of the cell. In some embodiments, the genome is of a cell of the same type or species as the modified cell. In some embodiments, the fragment locations are known to the reader. In some embodiments, identifying comprise locating the fragments with the received sequences.

In some embodiments, the reading further comprises reading an identifier. In some embodiments, an identifier is read in a plurality of fragments. In some embodiments, an identifier is read in all fragments. In some embodiments, the reading an identifier is performed between the identifying fragments and correcting. In some embodiments, reading the fragments comprises correcting an indel, point mutation or both. In some embodiments, identifiers are corrected by means of a decoder. In some embodiments, the reader comprises the coding matrix of the identifiers. In some embodiments, the identifiers and information have different coding matrixes. In some embodiments, the identifier is read separately from the stored information. In some embodiments, the identifier is decoded separately from the information. In some embodiments, the reading comprises maximum likelihood decoding. In some embodiments, maximum likelihood decoding ensures all ordinal numbers are represented in the identifiers. In some embodiments, all ordinal numbers are all numbers up to the total number of identifiers. In some embodiments, all ordinal numbers are all numbers up to the total number of fragments. For example, if there are 20 fragments each with an identifier than the fragments must be assigned the numbers 1 through 20 (or the ordinals 1^{st} through 20^{th}). The maximum likelihood decoding ensures that each number is represented once in the identifiers. This is done by selecting the identifier most likely to be each ordinal number. This can include starting with the ordinal number with the lowest probability of being encoded by a particular identifier and then progressing though the ordinals by increasing probability. The probability is determined by the error correcting code decoder. The use of maximum probability decoding greatly improves the overall performance of the method as it guarantees are ordering of the fragments. Coupled with the separate coding of the identifiers to improve fidelity, these two aspects greatly improve the overall quality of the received message and allow for longer storage times then methods previously known in the art.

In some embodiments, the reading further comprises ordering the fragments based on the read identifiers. In some embodiments, the reading further comprises producing an order of the fragments based on the read identifiers. In some embodiments, extracting is in the order of the fragments. In some embodiments, the method further comprises combining all extracted information into the stored information. In some embodiments, the combining is combining in order. In some embodiments, the method further comprises removing the identifiers from the fragments. In some embodiments, the method further comprises removing the identifiers from the information. In some embodiments, the combining comprises concatenating the fragments in order. In some embodiments, the order is established by the read identifiers.

In some embodiments, the identifiers are read/decoded separately. In some embodiments, the identifiers are read/decoded together. In some embodiments, the identifiers are read/decoded together for the maximum likelihood of all identifiers, such that there must be at least one identifier for each numerical value of the fragments. In some embodiments, the decoding of the identifiers is a maximum likelihood decoding. In some embodiments, the likelihood is determined by the error correcting code decoder.

By another aspect, there is provided a system comprising at least one hardware processor; and a non-transitory computer-readable storage medium having stored thereon program code, the program code executable by the at least one hardware processor to perform a method of the invention.

By another aspect, there is provided a computer program product comprising a non-transitory computer-readable storage medium having program code embodied therewith, the program code executable by at least one hardware processor to perform a method of the invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Example 1: The Noisy Shuffling Sampling Channel

The channel model discussed herein was initially presented in Shomorany and Heckel (referenced hereinabove) and is referred to as the Noisy Shuffling Sampling Channel. Originally, Shomorany and Heckel, applied the Noisy Shuffling Sampling Channel to the case of artificial DNA strand storage in vitro. Hereinbelow it is applied to actual in vivo living organisms, which will affect the actual parameters of the model.

Shomorany and Heckel considered the asymptotic regime of infinitely many microorganisms (DNA strands, or M which may grow to infinity, in their notations) and infinitely large DNA sequence length (DNA strand length, or L which may grow to infinity, in their notations).

The channel model is as follows. Data is written on M synthesized microorganisms (or DNA sequences) by the transmitter, where each synthesized microorganism is represented as a sequence of bits. The number of bits written on each microorganism is L. After a certain storage time, the synthesized microorganisms are read by the receiver, who wishes to reconstruct the original information stored onto all microorganisms. The biological medium, which is referred to as the Noisy Shuffling Sampling Channel, may introduce errors in the time between writing and reading. Notice that both channel inputs and output are over the binary alphabet (FIG. 1).

First, a portion of the microorganisms may not survive and thus is erased, depicted in FIG. 1 as microorganism erasure. This may happen due to fitness degradation of some of the microorganisms, which occurred by the code of the invention which changed their original content. Second, no particular spatial ordering of the microorganisms is kept over time. Therefore, the surviving microorganisms are read in random order with respect to their original order at the transmitter's hands. This phenomenon is captured in FIG. 1 as a permutation. Lastly, the channel imposes corruption on specific bits, meaning that some individual bits may be substituted (flipped) between the two parties. This may happen due to mutations at the single nucleotide level, depicted as nucleotide substitution in FIG. 1.

The data stored over the channel is a binary vector of =M·L bits divided into M binary vectors of length L each, which we call a message block. Each message block is stored over a different microorganism. After some storage time, the receiver reads the survived (non-erased) message blocks. Given that N microorganisms had survived, we end up with a binary vector of n=N·L bits.

The channel input is x=x₀ . . . xₘ₋₁=X₀ . . . X_{M-1}, which is a sequence of m=|x| bits (denoted by small letters xᵢ), or a sequence of M message blocks (denoted by capital letters Xᵢ), defined as a binary vector of L bits, where m=M·L. The message block Xᵢ corresponds to the data written onto microorganism number i, i∈{0, . . . , M−1}. The channel input alphabet is the binary alphabet{0,1}, meaning that xᵢ∈{0,1}, Xᵢ∈{0,1}^{L}.

The channel output is y=y₀ . . . yₙ₋₁=Y₀ . . . Y_{N-1}, which is a sequence of n=|y| bits (denoted by small letters yᵢ), or alternatively a sequence of N message blocks (denoted by capital letters Yᵢ), each of length L bits. The Noisy Shuffling Sampling Channel assumes that erasures occur at the entire message block level only (for instance, caused by the extinction of a whole microorganism), therefore also n=N·L. The channel output alphabet is the binary alphabet{0,1}, meaning that yᵢ∈{0,1}, Yᵢ∈{0,1}^{L}.

Next, a mathematical formulation of the channel model is assessed. The first phenomenon captured by the model, as seen in FIG. 1, is a constant erasure probability equal to Pₑ, meaning that each message block Xᵢ is erased with probability Pₑ and survives with probability 1−Pₑ, independently of all other message blocks {Xⱼ}_{j≠i}N is the (random) number of surviving (non-erased) message blocks, defined as the number of output message blocks

$$N = \frac{\vert y \vert}{L} .$$

Clearly, N is a sum of M idd Bernoulli random variables, each with a parameter 1−Pₑ, and this is a binomial random variable N˜B(M, 1−Pₑ).

The second phenomenon captured by the model, as seen in FIG. 1, is a permutation of the surviving message blocks. The microorganisms are numbered from 0 to M−1 when the iₜₕ message block is stored over the iₜₕ microorganisms. Given that the number of surviving microorganisms is N, the permutation vector π is defined as a random injective function from the set{0, . . . , N−1} to the set{0, . . . , M−1}, chosen uniformly at random from the set Π_{N}, which is the set of all injective functions from the set{0, . . . , N−1} to the set {0, . . . , M−1} (alternatively, Π_{N} is the set of all N-permutations of the set{0, . . . , M−1}). For instance, if M=3,N=2, then Π_{N}={{(0→0), (1→1)}, {(0→0), (1→2)}, {(0→1), (1→0)}, {(0→1), (1→2)}, {(0→2), (1→0)}, {(0→2), (1→1)}}, and is one of Π_{N} members. The number of N-permutations of {0, . . . , M−1} is

$$\vert {Π}_{N} \vert = \frac{M !}{( M - N ) !} ,$$

$$thus$$

$$π ∼ U [ \vert {Π}_{N} \vert = \frac{M !}{( M - N ) !} ]$$

(discrete probability distribution).

The last phenomenon captured by the model, as seen in FIG. 1, is a constant bit substitution probability equal to Pₛ, meaning that each output bit yᵢ has suffered from substitution with probability Pₛ, independently of all other bits {yⱼ}_{j≠i}. Let s=s₀ . . . . sₙ₋₁=S₀ . . . . S_{N-1} be the random binary vector corresponding to the individual bit substitutions pattern sᵢ˜Ber(Pₛ) in an iid manner.

Finally, one can write Yᵢ=X_{π(i)}⊕Sᵢ, i=0, . . . , N−1, ⊕ denotes the element-wise XOR operation, meaning that each channel output message block is a corrupted version of one of the input message blocks if the latter survived.

Before quoting the main result in Shomorany and Heckel, which is the Noisy Shuffling Sampling Channel capacity in the infinite block-length regime of M, L→∞, the channel capacity is quantified in the present storage scenario.

An (M, L) microorganisms storage code C, is defined as a set of |C| codewords of the form x=x₀ . . . xₘ₋₁=X₀ . . . X_{M-1}, where each codeword is a binary vector of length M·L bits, or equivalently as M message blocks of length bits each, together with an encoding function f: {0, . . . , |C|−1}→C from the possible messages to their corresponding channel inputs. A decoding function is a mapping h: {0,1}^{N·L→{}0, . . . , |C|−1}, which accepts the channel output y=y₀ . . . yₙ₋₁=Y₀ . . . . Y_{N-1} and assigns it with one of the possible messages {0, . . . , |C|−1}. The corresponding coding rate is

$$R = \frac{log \vert C \vert}{m} = \frac{log \vert C \vert}{ML} ,$$

which is the total number of information bits stored relative to the available channel resources (for instance, the total number of codons).

Reliable storage is defined as a vanishing decoding error probability as the

$$Pr ( h ( y ) \neq x ) {\to }^{M , L \to ∞} 0.$$

codewords' length is growing to infinity, i.e., Reliable storage is possible whenever R<C*, where C*C* is the classic channel capacity defined as

$${C}^{*} = {sup}_{Pr ( x )} I ( x ; y ) , and I ( x ; y ) = E [ - log \frac{p ( x , y )}{p ( x ) p ( y )} ]$$

is the mutual information between the channel input and output. The supremum is taken over all possible input distributions. In Shomorany and Heckel, the authors derived C* for the Noisy Shuffling Sampling Channel in the asymptotic regime of M, L→∞. Their result is quoted in the following theorem.

Theorem 1. The capacity of the noisy shuffling-sampling channel is

$${C}^{*} = ( 1 - {P}_{e} ) ( 1 - H ( {P}_{s} ) - \frac{1}{β} ) \qquad ( 1 )$$

as long as

$${P}_{s} < 0.25 , 1 - H ( 2 {P}_{s} ) - \frac{2}{β} > 0.$$

In particular, if β≤1 then the capacity is C*=0. Here,

$$β = \frac{L}{log M} ,$$

and H(t)=−t log t−(1−t) log(1−t) is the binary entropy function.

This result gives an upper bound on the achievable coding rate of any coding scheme over the Noisy Shuffling Sampling Channel model in the asymptotic regime, thus also an upper bound of the achievable coding rate of any (M, L) microorganisms storage code in the non-asymptotic regime of M, L→∞.

### Example 2: Writing Process

A chunk of k information bits, generated by an information source is stored over the living organism's DNA by the transmitter. The storage will change the DNA contents of the organism, where M different synthesized DNA sequences will be written onto M microorganisms, altogether carrying the information message.

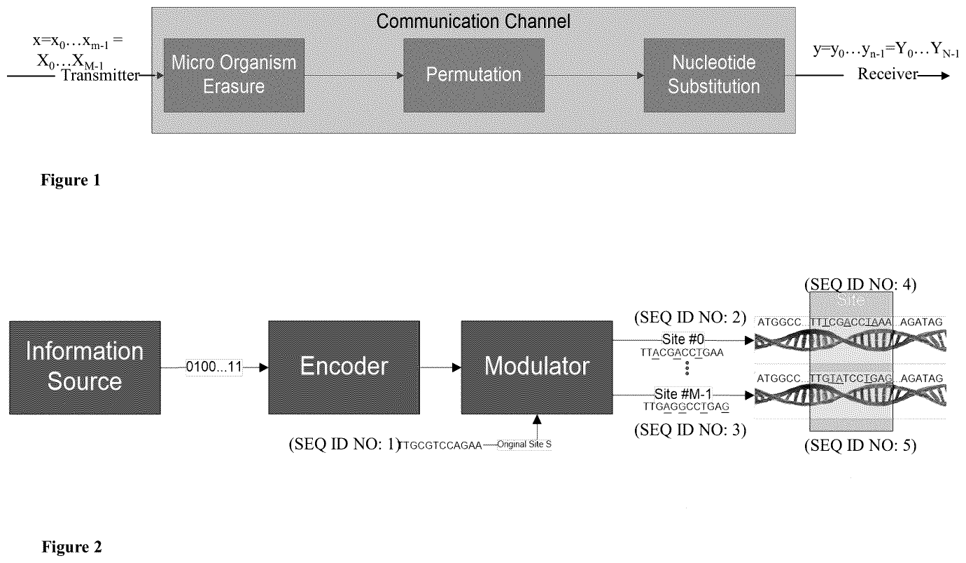

The Encoder, discussed hereinbelow, encodes the information bits into M message blocks, binary vectors of length L bits each, using some error correction code. The overall encoder output is a binary vector of m=M·L bits. Next, the Modulator changes the DNA sequence of the iₜₕ microorganism (i∈{0, . . . , M−1}) in a specific area we call a site that carries the information of the iₜₕ iₜₕ message block created by the Encoder. A site S is defined as a sequence (string) of 3|S| nucleotides (or |S | codons) at a given and fixed location among all the microorganisms' DNA sequences, as depicted in the box in FIG. 2. It should be emphasized that one starts with a single microorganism with a DNA sequence and creates M different versions of it by changing its content among the site S. The overall modulator output is M synthesized sites.

Therefore, the writing (or synthesis) process involves overwriting M parallel DNA sequences of a living organism, starting at a given and fixed position among all of them, with a length of 3|S| nucleotides. Only specific nucleotides (underlined ones in FIG. 2) are changed at each site compared to the original sequence and are bearing the information stored over the living organism in a way that will be explained hereinbelow.

### Example 3: Living Organism Channel

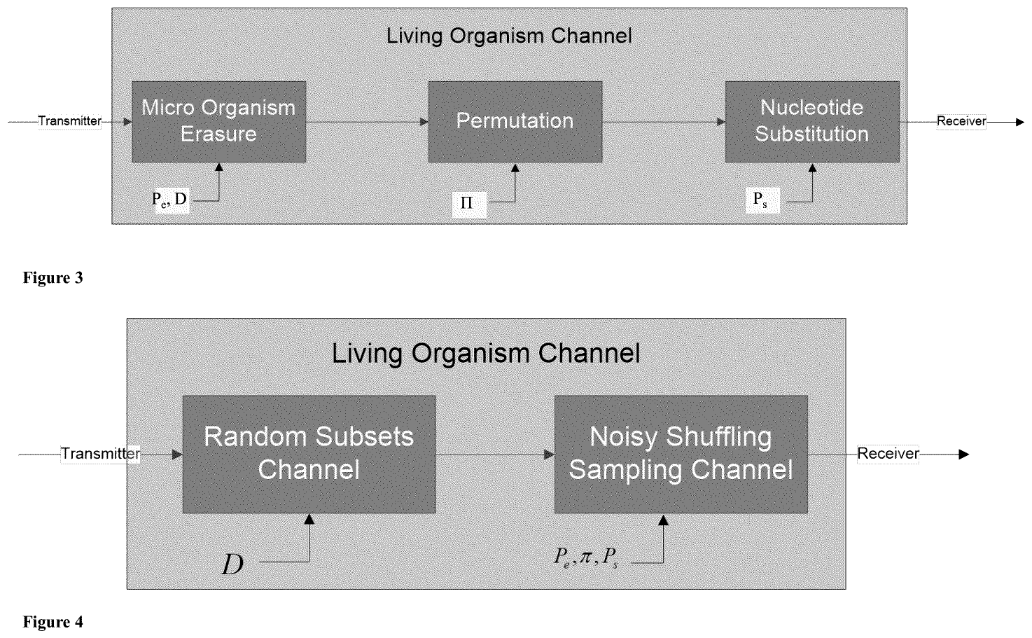

The synthesized microorganisms are carrying the programed information for some time. The biological medium, which is referred to as the Living Organism Channel, may introduce errors during the time between writing and reading according to evolution, as depicted in FIG. 3.

The first biological phenomenon captured in the Living Organism Channel is that a portion of the microorganisms may not survive and thus are erased, depicted in FIG. 3 as microorganism erasure. This may happen due to two main reasons, which are captured by the parameter Pₑ and a dictionary D (FIG. 3). The first reason is fitness degradation of some of the microorganisms, a consequence of the coding, which changed their original genetic content and maybe overloaded the organism with too many changes that decreased the growth rate, or fitness of the microorganism. This behavior is modeled by a globally constant microorganism erasure probability equal to Pₑ, and later there is presented a method for estimating this erasure probability. The second reason for a microorganism erasure is disallowed changes of codons, which is defined as an accidental change of a codon crucial to the evolution of the microorganism. The simplest example may be a non-synonymous codon change, causing a protein change. From now on, it is assumed that nature holds a dictionary of such allowed changes for each organism, D, which is a mapping function from each codon location in the site S in the organism's DNA, to a set of allowed codons. The dictionary D dictates the constraint that each specific codon in each particular location in the DNA is allowed to be changed at a particular set of codons. Any other change (i.e., a codon change into another codon which is not a member of the set mapped by D) will cause a microorganism's death and, finally, an erasure of the relevant DNA sequence. Hereinbelow is provided an estimate of dictionary D and how to design a code not violating those constraints. It is also assumed that only the transmitter holds D, but not the receiver. The reason for such an assumption is that in a practical storage application, the encoder has computation resources and are operating offline on a computer, while the decoder may be implemented in vivo over some organism and operating in real time. Therefore, an appropriate coding strategy is needed.

The second phenomenon captured by Living Organism Channel is that no particular spatial ordering of the microorganisms is kept over time. Therefore, the surviving microorganisms are read in random order with respect to their original order at the transmitting party's hands. This phenomenon is captured in FIG. 3 as permutation and is caused mainly because of the current reading technologies, in which, each time, a random DNA sequence is read from all available sequences.

Lastly, the channel imposes corruption on specific nucleotides, meaning that some individual nucleotides may be substituted between the two encoding and decoding. This may happen due to mutations at the single nucleotide level, depicted as nucleotide substitution in FIG. 3. It is assumed that all nucleotides may substitute with probability of Pₛ, meaning that the original nucleotide may substitute into any one of the three other possible nucleotides with equal probability of

$$\frac{{P}_{s}}{3} ,$$

and overall may remain unchanged with probability 1−Pₛ. Hereinbelow a method for estimating this substitution probability is provided.

The Living Organism channel may be presented as the Noisy Shuffling Sampling Channel, preceded by the Random Subsets Channel, which is defined hereinbelow, constraining the synthesized microorganisms to follow the allowed codon changes according to the dictionary D (FIG. 4). Pₑ, Pₛ are the parameters of the Noisy Shuffling Sampling Channel as discussed hereinabove. It is the random permutation and is not an actual parameter of the model which can be estimated apriori.

### Example 4: Reading Process

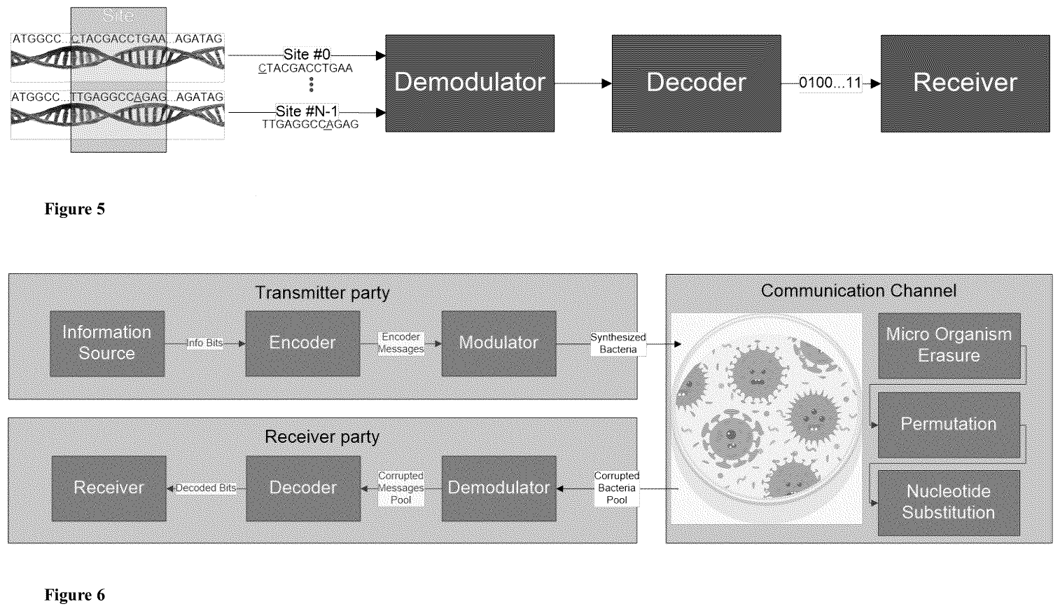

After a certain period, the receiver wishes to read the stored information on the synthesized microorganisms. The reading process, depicted in FIG. 5, starts with reading the microorganisms' sites which are known to both the transmitter and the receiver. As illustrated in FIG. 5, some nucleotides substitutions may occur (depicted as underlined letters), a permutation occurs (for example, microorganism number0 0 upon writing is read as microorganism number N−1), and some microorganisms may be erased, if N<M.

The Demodulator retranslates each read site into an encoded (and maybe corrupted) message block (a binary vector of length L) in a way that will be explained hereinbelow. Next, the decoder sorts the permuted message blocks, applies error correction, and reconstructs the k information bits.

The overall system is depicted in FIG. 6. The “Pool” terminology is used to emphasize the loss of microorganisms' spatial ordering at the receiver.

### Example 5: Finite Block-Length Analysis of the Noisy Shuffling Sampling Channel

Next, the Finite Block-Length capacity of the Noisy Shuffling Sampling Channel is derived.

The goal here is to analyze the actual realistic case of the non-asymptotic regime. Since current synthetic biology technologies limit the number of microorganisms that can be synthesized in parallel (M), and also the length of synthesized nucleotides sequences in each microorganism (L) to the order of a few hundreds. For this purpose, the finite block length (FBL) analysis will be used. It is briefly summarized hereinbelow.

Consider a communication channel with input x and output y, both over some alphabets, with some probability distribution Pr(y|x). The following theorem gives a lower bound on the achievable coding rate of any coding scheme over this communication channel, under some allowed decoding error probability.

Theorem 2. For any distribution Pr(x) on the input alphabet, there exists a code C with |C| codewords and an average probability of error ε at most

$$ε \leq E [ exp ( - {[ i ( x ; y ) - log \frac{\vert C \vert - 1}{2} ]}^{+} ) ] . \qquad ( 2 )$$

$$Here ,$$

$$i ( x ; y ) = log \frac{Pr ( y \vert x )}{Pr ( y )} = log \frac{Pr ( x \vert y )}{Pr ( x )} , \qquad ( 3 )$$

is the information density between the channel input and output, which is a function of the random variables x, y. Therefore, i(x; y) is also a random variable by itself. Also, the expectation is over the joint distribution of x, y.

The above result (called the Dependence Testing (DT) bound) provides a lower bound on the codebook size |C|, which is also a lower bound on the achievable coding rate of an (M, L) microorganisms storage code C, which is defined by

$$R = \frac{log \vert C \vert}{m} \qquad ( 4 )$$

The storage reliability in the DT bound is quantified by ε, the average probability of a decoding error, indicating the average fraction number of situations in which the decoder fails to reconstruct the original message. Here, the codewords' length is kept constant and not growing asymptotically; therefore, unlike Shannon's original channel capacity theorem, the metric ε must be taken into account.

Next, Theorem 2 is applied to the Noisy Shuffling Sampling Channel, introduced hereinabove. The channel's input x is a binary vector of length m=M·L consisting of M message blocks of length L bits, each one stored on a distinct microorganism, indexed by 0 to M−1. The N surviving organisms (after the erasures) are permuted randomly, modeled by a random injective function n from the set {0, . . . , N−1} to the set {0, . . . , M−1}. Lastly, each bit in the N surviving messages is randomly flipped in an iid manner with probability Pₛ. This results in the channel's output yy, which is a binary vector of length n=N·L.

To apply Theorem 2, an expression for the information density i(x; y) must be derived. First, a lower bound on Pr(x|y) is derived, which is the inverse conditional channel distribution. Note that Pr(x|y)is a random variable which is a function of the random variables x, y.

Lemma 1. The conditional probability Pr(x|y) of the Noisy Shuffling Sampling Channel satisfies

$$Pr ( x \vert y ) = \frac{( M - N ) !}{M !} {2}^{- ML} {( 2 - 2 {P}_{s} )}^{NL} {\sum }_{π ∈ {\prod }_{N}} {( \frac{{P}_{s}}{1 - {P}_{s}} )}^{d ( {x}_{π} , y )} , \qquad ( 5 )$$

Where d(x, y) is the hamming distance between x_{π} and yx_{π} is the vector obtained by permuting x by π, i.e., if x=X₀ . . . X_{M-1} then x_{π}=X_{π(0)} . . . X_{π(N-1)}, and

$$N = \frac{\vert y \vert}{L} .$$

Proof. Recall from above that Yᵢ=X_{π(i)}⊕Sᵢ, i=0, . . . , N−1 , where s=S₀ . . . Sₙ₋₁=S₀ . . . S_{N-1} is the random binary vector that corresponds to the individual bit substitutions pattern and Sᵢ is a vector of L Bernoulli random variables with parameter Pₛ. Then, given y, the conditional distribution Pr(x|y) satisfies

$$Pr ( x \vert y ) {=}^{( i )} Pr ( x \vert y , N ) {=}^{( ii )} {\sum }_{π ∈ {\prod }_{N}} Pr ( π \vert y , N ) Pr ( x \vert y , N , π ) {=}^{( iii )} {\sum }_{π ∈ {\prod }_{N}} Pr ( π \vert y , N ) \text{} {\prod }_{i = 0}^{M - 1} Pr ( {X}_{i} \vert y , N , π ) {=}^{( iv )} {=}^{( iv )} {\sum }_{π ∈ {\prod }_{N}} Pr ( π \vert N ) ( {\prod }_{i = 0}^{M - N - 1} {\frac{1}{2}}^{L} ) ( {\prod }_{i = 0}^{N - 1} Pr ( {X}_{π ( i )} \vert y ) ) = {2}^{- ( M - N ) L} {\sum }_{π ∈ {\prod }_{N}} Pr ( π \vert N ) {\prod }_{i = 0}^{N - 1} Pr ( {X}_{π ( i )} ⊕ {Y}_{i} \vert y ) {=}^{( v )} {=}^{( v )} {2}^{- ( M - N ) L} \frac{( M - N ) !}{M !} {\sum }_{π ∈ {\prod }_{N}} {\prod }_{i = 0}^{N - 1} Pr ( {X}_{π ( i )} ⊕ {Y}_{i} = {S}_{i} ) {=}^{( vi )} {2}^{- ( M - N ) L} \text{} \frac{( M - N ) !}{M !} \text{} {\sum }_{π ∈ {\prod }_{N}} ( {\prod }_{i = 0}^{NL - 1} {{P}_{s}^{{s}_{i}} ( 1 - {P}_{s} )}^{1 - {s}_{i}} ) == \frac{( M - N ) !}{M !} {2}^{- ML} {( 2 - 2 {P}_{s} )}^{NL} {\sum }_{π ∈ {\prod }_{N}} {( \frac{{P}_{s}}{1 - {P}_{s}} )}^{{\sum }_{i = 0}^{NL - 1} {s}_{i}} {=}^{( vii )} \frac{( M - N ) !}{M !} {2}^{- ML} {( 2 - 2 {P}_{s} )}^{NL} {\sum }_{π ∈ {\prod }_{N}} {( \frac{{P}_{s}}{1 - {P}_{s}} )}^{d ( {x}_{π} , y )}$$

(i) follows from the fact that

$$N = \frac{\vert y \vert}{L} .$$

(ii) follows from the law of total probability over π. (iii) results from the fact that given π, x depends only on the random substitutions pattern vector ss, and in particular {Xᵢ} are iid distributed. Also given πx can be divided into a set of M−N erased message blocks and a set of the remaining N non erased messages. (iv) follows since no information is available from the first group, imposing probability equal to

$${\frac{1}{2}}^{( M - N ) L} .$$

Next, πis uniformly distributed over ΠN (v). In (vi), we plugged the distribution of s=S₀ . . . S_{N-1}, Sᵢ=X_{π(i)}⊕Yᵢ, i=0, . . . , N−1. (vii) follows since by definition, d(x_{π}y)=Σᵢ₌₀^{NL-1}sᵢ.

To calculate i(x; y), one needs to derive an expression for the distribution of the random variable

$${\sum }_{π ∈ {\prod }_{N}} {( \frac{{P}_{s}}{1 - {P}_{s}} )}^{d ( {x}_{π} , y )} ,$$

which is a function of the random hamming distance d(x_{π}, y) for any π. Intuitively, assuming the channel chose the permutationπ, the expression

$${\sum }_{π ∈ {\prod }_{N}} {( \frac{{P}_{s}}{1 - {P}_{s}} )}^{d ( {x}_{π} , y )}$$

will be dominated by the term defined by the permutation πΠ, since this permutation minimizes the hamming weight d(x_{π}, y). The following lemma makes this intuition more formal.

Lemma 2. The conditional distribution of the noisy shuffling sampling channel can be written as

$$Pr ( x \vert y ) = \frac{( M - N ) !}{M !} {2}^{- ML} {( 2 - 2 {P}_{s} )}^{NL} {( \frac{{P}_{s}}{1 - {P}_{s}} )}^{T} + A , \qquad ( 6 )$$

Where T˜B(NL, Pₛ) is a binomial random variable and A A is some nonnegative random variable.

Proof. Let π^{*} be the random permutation chosen by the channel, therefore Yᵢ=X_{π*(i)}⊕Sᵢ, i=0, . . . , N−1.

By Lemma 1, one can write

$$Pr ( x \vert y ) = \frac{( M - N ) !}{M !} {2}^{- ML} {( 2 - 2 {P}_{s} )}^{NL} {\sum }_{π ∈ {Π}_{N}} {( \frac{{P}_{s}}{1 - {P}_{s}} )}^{d ( {x}_{π} , y )} = \frac{( M - N ) !}{M !} {2}^{- ML} {( 2 - 2 {P}_{s} )}^{NL} ( {( \frac{{P}_{s}}{1 - {P}_{s}} )}^{d ( {x}_{{π}^{*}} , y )} + {\sum }_{π ∈ {Π}_{N} ∖ {π}^{*}} {( \frac{{P}_{s}}{1 - {P}_{s}} )}^{d ( {x}_{π} , y )} ) = \frac{( M - N ) !}{M !} {2}^{- ML} {( 2 - 2 {P}_{s} )}^{NL} {( \frac{{P}_{s}}{1 - {P}_{s}} )}^{d ( {x}_{{π}^{*}} , y )} + A$$

where A is a nonnegative random variable. Define the function 1{Z}, where Z is a message block, as the sum of indicator functions of Z's elements, namely 1{Z}=Σᵢ₌₀^{l-1}1{zᵢ=1}. 1{Z} is also the hamming weight of Z. Now, one notices that

$$d ( {x}_{{π}^{*}} , y ) {=}^{( i )} {\sum }_{i = 0}^{N - 1} 1 \{ {X}_{{π}^{*} ( i )} ⊕ {Y}_{i} \} {=}^{( ii )} {\sum }_{i = 0}^{N - 1} 1 \{ {X}_{{π}^{*} ( i )} ⊕ ( {X}_{{π}^{*} ( i )} ⊕ {S}_{i} ) \} = {\sum }_{i = 0}^{N - 1} 1 \{ {S}_{i} \} = {\sum }_{i = 0}^{N - 1} {\sum }_{j = 0}^{L - 1} 1 \{ {s}_{j + iL} = 1 \} {∼}^{( iii )} B ( NL , {P}_{s} ) ,$$

where (i) follows from the definition of the hamming weight, (ii) from the connection Yᵢ=X_{π(i)}^{*}⊕Sᵢ, i=0, . . . , N−1, and (iii) from the fact that {sᵢ} are iid Bernoulli random variables Ber(Pₛ) and the sum of NL iid Bernoulli Ber(Pₛ) is a Binomial random variable B (NL, Pₛ).

Now one can explicitly derive the DT bound (Theorem 2) for the Noisy Shuffling Sampling Channel.

Lemma 3. The DT bound (Theorem 2) for the Noisy Shuffling Sampling Channel is given by

$$ε \leq {\sum }_{r = 0}^{M} {\sum }_{t = 0}^{rL} ( \begin{matrix}M \\ r\end{matrix} ) {( 1 - {P}_{e} )}^{r} \text{} {P}_{e}^{M - r} ( \begin{matrix}rL \\ t\end{matrix} ) min \{ {{P}_{s}^{t} ( 1 - {P}_{s} )}^{rL - t} , {2}^{k - 1} \frac{M !}{( M - r ) !} {2}^{- rL} \} ,$$

for codebook size |C|=2^{k} and rate

$$R = \frac{log \vert C \vert}{m} = \frac{k}{m} .$$

Proof. Let ||C|=2^{k} be the size of the code, then by Theorem 2

$$ε \leq E [ {2}^{- {[ i ( X ; Y ) - log \frac{{2}^{k} - 1}{2} ]}^{+}} ] \approx E [ {2}^{- {[ i ( X ; Y ) + 1 - k ]}^{+}} ] .$$

Now, using Lemma 2, we have

$$E [ {2}^{- {[ i ( x ; y ) + 1 - k ]}^{+}} ] = E [ {2}^{- max \{ 0 , i ( x ; y ) + 1 - k \}} ] {=}^{( i )} {\sum }_{r = 0}^{M} {\sum }_{t = 0}^{rL} Pr ( N = r ) Pr ( T = t \vert N = r ) {2}^{min \{ 0 , k - 1 - i ( x , y ) \}} = {\sum }_{r = 0}^{M} {\sum }_{t = 0}^{rL} ( \begin{matrix}M \\ r\end{matrix} ) {( 1 - {P}_{e} )}^{r} {P}_{e}^{M - r} ( \begin{matrix}rL \\ t\end{matrix} ) {{P}_{s}^{t} ( 1 - {P}_{s} )}^{rL - t} min \{ 1 , {2}^{k - 1} \frac{Pr ( x )}{Pr ( x \vert y )} \} {\leq }^{( ii )} {\leq }^{( ii )} {\sum }_{r = 0}^{M} {\sum }_{t = 0}^{rL} ( \begin{matrix}M \\ r\end{matrix} ) {( 1 - {P}_{e} )}^{r} {P}_{e}^{M - r} ( \begin{matrix}rL \\ t\end{matrix} ) {{P}_{s}^{t} ( 1 - {P}_{s} )}^{rL - t} min \{ 1 , {2}^{k - 1} \frac{{2}^{- ML}}{\frac{( M - r ) !}{M !} {2}^{- ML} {( 2 - 2 {P}_{s} )}^{rL} {( \frac{{P}_{s}}{1 - {P}_{s}} )}^{t}} \} \leq \leq {\sum }_{r = 0}^{M} {\sum }_{t = 0}^{rL} ( \begin{matrix}M \\ r\end{matrix} ) {( 1 - {P}_{e} )}^{r} {P}_{e}^{M - r} ( \begin{matrix}rL \\ t\end{matrix} ) {( 1 - {P}_{s} )}^{rL} {( \frac{{P}_{s}}{1 - {P}_{s}} )}^{t} min \{ 1 , {2}^{k - 1} \frac{M !}{( M - r ) !} {( 2 - 2 {P}_{s} )}^{- rL} {( \frac{{P}_{s}}{1 - {P}_{s}} )}^{t} \} \leq \leq {\sum }_{r = 0}^{M} {\sum }_{t = 0}^{rL} ( \begin{matrix}M \\ r\end{matrix} ) {( 1 - {P}_{e} )}^{r} {P}_{e}^{M - r} ( \begin{matrix}rL \\ t\end{matrix} ) min \{ {{P}_{s}^{t} ( 1 - {P}_{s} )}^{rL - t} , {2}^{k - 1} \frac{M !}{( M - r ) !} {2}^{- rL} \}$$

where (i) follows from the dependency of the information density on the random variables N,T, and (ii) from Lemma 2 and the nonnegativity of the random variable A.

From now on, a code whose average error probability satisfies the DT bound (Lemma 3) is referred to as an (M, L, ε) microorganisms storage code C_{FBL} with |C_{FBL} | codewords, and rate

$${R}_{FBL}^{*} = \frac{log \vert {C}_{FBL} \vert}{m} = \frac{k}{m} \qquad ( 8 )$$

The channel capacity of the Noisy Shuffling Sampling Channel, in the non-asymptotic regime of M microorganisms of length L and m=ML, which we denote by C^{*}_{FBL}, is therefore guaranteed to be bounded by R^{*}_{FBL}≤C^{*}_{FBL}≤C^{*}, where the upper bound is given by Theorem 1 and the lower bound by Lemma 3.

The main result of all this is summarized in the following Theorem.

Theorem 3. The channel capacity of the Noisy Shuffling Sampling Channel, in the Finite Block-Length regime of M microorganisms of length L each such that m=ML, denoted by C^{*}_{FBL}, is bounded by

$$\frac{{k}^{*}}{m} \leq {C}_{FBL}^{*} \leq ( 1 - {P}_{e} ) ( 1 - H ( {P}_{s} ) - \frac{log M}{L} ) \qquad ( 9 )$$

$$Where$$

$${k}^{*} = max \{ k : ε \leq E [ {2}^{- {[ i ( x ; y ) + 1 - k ]}^{+}} ] \} = max \{ k : ε \leq {\sum }_{r = 0}^{M} {\sum }_{t = 0}^{rL} ( \begin{matrix}M \\ r\end{matrix} ) {( 1 - {P}_{e} )}^{r} {P}_{e}^{M - r} ( \begin{matrix}rL \\ t\end{matrix} ) min \{ {{P}_{s}^{t} ( 1 - {P}_{s} )}^{rL - t} , {2}^{k - 1} \frac{M !}{( M - r ) !} {2}^{- rL} \} \} \qquad ( 10 )$$

Proof. It was already shown that C^{*}_{FBL} is upper bounded by Theorem 1 and lower bounded by Lemma 3. First, the actual value of

$${C}^{*} = ( 1 - {P}_{e} ) ( 1 - H ( {P}_{s} ) - \frac{log M}{L} )$$

was plugged in. Second, in the highest possible rate from Lemma 3 was plugged, which is the maximal value of k such that the right-hand side of the DT bound (Theorem 2) is larger than ε.

### Example 6: Bounds Evaluation

Numerical simulations were performed to evaluate the FBL capacity of the channel. The parameters M=180, L=144, ε=10⁶ were fixed and the parameters Pₑ, Pₛ were varied.

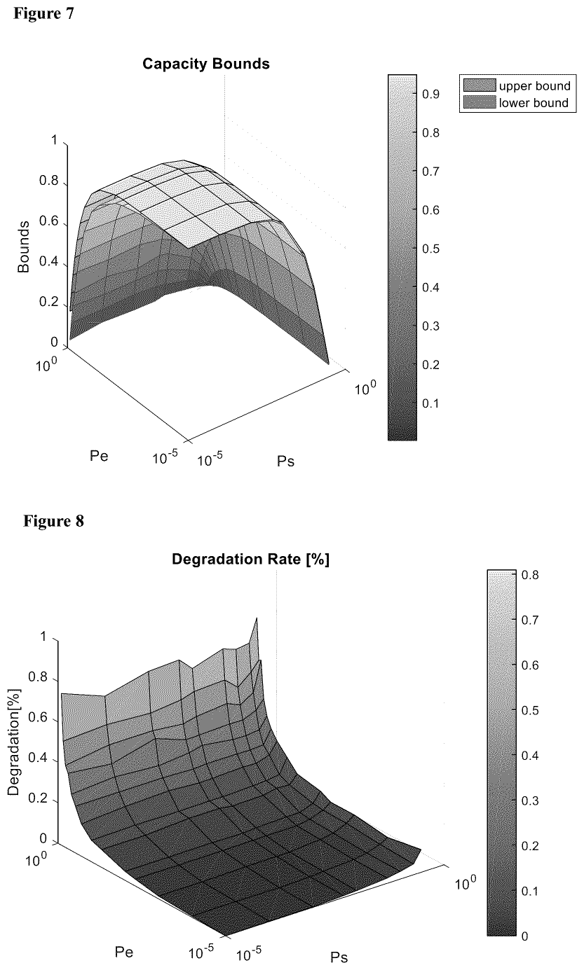

As can be seen in FIG. 7, the upper bound is the classic capacity C^{*}, and also the upper bound in equation (9), and the lower bound is the achievable coding rate R^{*}_{FBL} and also the lower bound in equation (9).

Next, the normalized degradation rate, defined as the relative gap between the lower and higher bound,

$$D = \frac{{C}^{*} - {R}_{FBL}^{*}}{{C}^{*}} = 1 - \frac{{R}_{FBL}^{*}}{{C}^{*}} \qquad ( 11 )$$

was simulated. Intuitively, an erasure of a message causes more information loss than a single substitution. This is reflected in Error! Reference source not found. 8, a growing erasure probability causing a more substantial degradation in the achievable rate compared to a growing substitution probability. Therefore, for high erasure probability, the actual optimal coding rate may be significantly worse than the one implied by Theorem 1. Moreover, there are ML possible locations for substitutions errors. In contrast, there are only M possible erasures, as substitutions occur at a single nucleotide, and erasures occur for the whole microorganisms. This is another explanation for the mild gap between the two bounds along the substitutions axis, but not along the erasures axis-the actual codeword length for the erasures part in the entire error mechanism is much smaller than that of the substitutions; therefore, the degradation caused by growing erasure probability is much more significant.

### Example 7: Random Subsets Channel

Motivated by the Living Organism Channel given hereinabove, a new communication channel called the Random Subsets Channel is provided and analyzed. The channel model assumes that nature holds a “dictionary” of allowed codon changes for each organism, which may be location dependent. In other words, in each codon in the DNA sequence of the organism, only specific codon changes are permitted by nature. The allowed changes are described by a dictionary that maps a particular location in the DNA into a set of allowed codons. Any change of a codon to a codon that is not one of the allowed changes would lead to the microorganism's death.

The simplest example for such a dictionary is the Amino Acid (AA) dictionary, allowing only synonymous (codon) changes. For instance, if the codon in the organism's DNA sequence is from the set L={CTT, CTC, CTA, CTG}, that includes all codons which translate into the AA “Leu”, then the allowed changes in that codon are changes to any of the codons from the set L.

The Random Subsets Channel generalizes the AA preservation constraint in the following ways. First, both the receiver and the transmitter may not know the dictionary (constraints or subsets) in advance. Second, the constraints can be arbitrary, as explained in more detail hereinbelow.

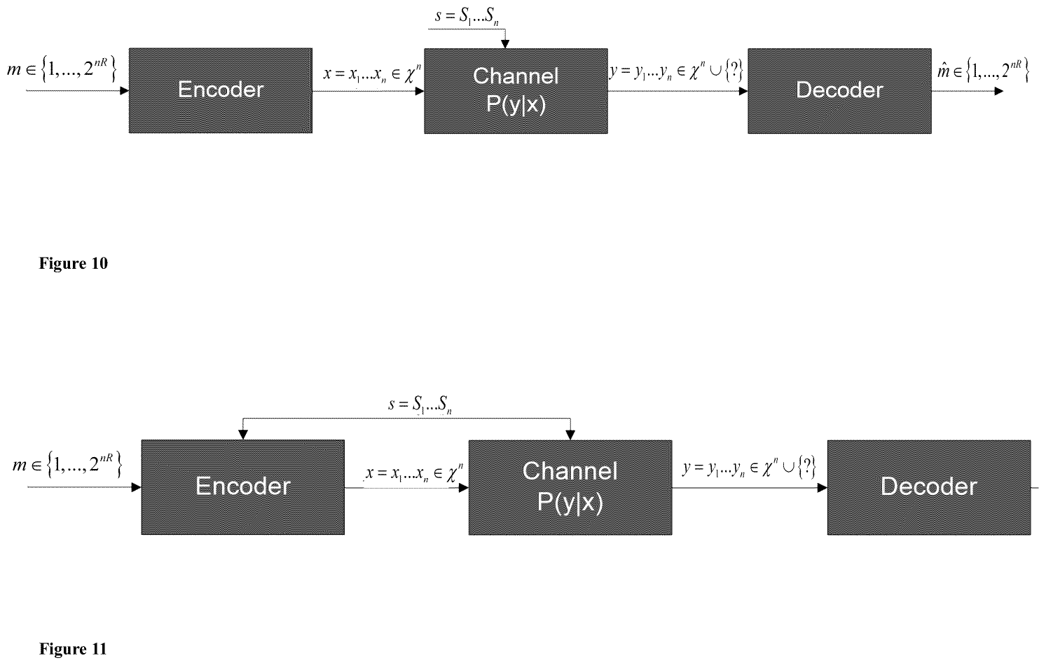

The Random Subsets Channel: Consider the Random Subset Channel, given in FIG. 10, defined by a discrete finite input alphabet x. For example, in the DNA storage setting, χ is the natural codons alphabet as the set of all |χ|=64 nucleotide triplets. Upon transmitting a vector x=(x₁, . . . , xₙ)∈χⁿx=(x₁, . . . , xₙ)∈χⁿ by n n channel usages, the channel output is the input vector x or?, where? represents a complete vector erasure. According to a probability distribution p(S), the channel, upon channel usage i, picks in an i.i.d fashion a subset Sᵢ⊂χ of size K. The subset Sᵢ represents the set of allowed symbols in the iₜₕ coordinate. The vector s=S₁. . . . Sₐ whose iₜₕ coordinate is the subset Sᵢ is called the state vector. The channel erases the vector x, i.e., it outputs?, if there is at least one index i such that xᵢ∉Sᵢ. Otherwise, the channel outputs x, namely the channel introduces no errors. Note that the state vector s may or may not be known to the encoder or the decoder in general. Information transmission is possible only if the encoder does not violate any subset constraint, i.e., xᵢ∈Sᵢ for any i; therefore, we assume that the encoder knows non-causally all the channel states {Sᵢ}ᵢ₌₁ⁿ and thus also satisfies all the subsets constraints. This assumption is motivated by the DNA setting since we assume that the genomic data and constraints are known in advance at the time of encoding. Hereinabove there was presented a method to estimate the state vector for a given organism.

The encoder maps one of the 2^{nR} possible messages, m, into the channel input x∈χⁿ, and the decoder maps a channel output y∈χⁿ∪{?}y∈χⁿ∪{?} into a decoded message m. The code rate is, therefore, R bits per channel use.

Next, several settings of the Random Subsets Channel were considered and analyzed.

Channel analysis: First, the simplest scenario was considered in which both the encoder and decoder know the state vector. The channel capacity in this setting is clearly C=log K since the channel boils down to a perfectly clean channel with input and output alphabets of size K. Indeed, the encoder and decoder can agree in advance on an injective mapping from the possible subsets Sᵢ of allowed symbols to the set of integers {1, . . . , K}. Therefore, in each channel usage, one can transmit log K bits.

Next, the more challenging scenario was considered, where only the encoder knows the state vectors, as shown in FIG. 11.

The capacity of a channel with side information at the encoder side is given by the well-known Gelfand-Pinsker (GP) Theorem, given next. Note that capital letter variables represent symbols: for example, S stands for a state symbol (a subset containing K unique elements), whereas s stands for the state vector.

Theorem 4. The capacity of a discrete, memoryless channel with a state S which is i.i.d distributed according to the probability distribution p(S) and is available non-causally at the encoder is

$${C}_{GP} = {{max}_{p ( U \vert S )}}_{X = f ( U , S )} ( I ( U ; Y ) - I ( U ; S ) ) {C}_{GP} = {{max}_{p ( U \vert S )}}_{X = f ( U , S )} ( I ( U ; Y ) - I ( U ; S ) ) \qquad ( 12 )$$

Where U is some random variable, and X=f(U,S) is a deterministic function of U, S. Also, the channel input alphabet is χ, the output alphabet is X∪{?}χ∪{?}, the state alphabet S is the set of all subsets of χ containing K distinct elements with

$$\vert S \vert = ( \begin{matrix}\vert χ \vert \\ K\end{matrix} ) ,$$

and the alphabet U of U holds

$$\vert U \vert \leq min \{ \vert χ \vert \vert S \vert , \vert χ \vert + \vert S \vert \} = \vert χ \vert + ( \begin{matrix}\vert χ \vert \\ K\end{matrix} ) \vert U \vert \leq min \{ \vert χ \vert \vert S \vert , \vert χ \vert + \vert S \vert \} = \vert χ \vert + ( \begin{matrix}\vert χ \vert \\ K\end{matrix} ) .$$

Without loss of generality, it is assumed that χ={1,2, . . . , [χ]}, and denote it by Sᵢ(j) the jₜₕ smallest element of the state Sᵢ for j∈{1, . . . , K}.

Next, two interesting cases of channel states probability distributions are considered. The first case is called the Partial Random Subsets Channel, it is a direct extension of the AA dictionary. In this case, a random variable Sᵢ is independently and uniformly distributed over a set of

$$\vert \frac{\vert χ \vert}{K}$$

subsets of χ, each of size K. This case models a genome in which mutations are DNA location independent; the allowed codon changes depend only on the codon itself and not on its location in the DNA. Mutation here means any change of codon.

In the second case, which is called the Completely Random Subsets Channel, the subsets are drawn independently and uniformly at random from all

$$( \begin{matrix}\vert χ \vert \\ K\end{matrix} )$$

possible subsets (of size K). This case models a genome in which mutations are location-dependent: the allowed changes of a codon depend both on the codon itself and its exact location in the DNA.

### Example 8: Partial Random Subsets Channel

In this channel model, the random variables is uniformly distributed over

$$\frac{\vert χ \vert}{K}$$

sets of size K K each, that form a partition of χ. i.e., there exist

$$\frac{\vert χ \vert}{K}$$

sets sᵢ, i∈

$$\{ 1 , … , \frac{\vert χ \vert}{K} \} {S}_{i} ,$$

$$\{ 1 , … , \frac{\vert χ \vert}{K} \}$$

of size K, such that x=∪ᵢ₌₁^{|v|/K}sᵢ∩sⱼ=ϕv=∪ᵢ₌₁^{|χ|/K}sᵢ, sᵢ∩sⱼ=ϕ for distinct i, j, and the channel state probability distribution is

$$p ( S = {s}_{i} ) = \frac{K}{\vert χ \vert} for i = 1 , … , \frac{\vert χ \vert}{K}$$

and zero otherwise. This alphabet partition (i.e., the support of the random variables) is unknown to the decoder. It models a genome in which the mutations are location-independent. Next, the capacity of this channel is derived.

Theorem 5. The Partial Random Subsets channel capacity is C=log K.

Proof. Since the encoder does not violate the subsets constraint, and the output alphabet size is K, then the capacity is at most log K. Next, it is shown that the capacity is at least log K.

By Theorem 4 and the fact that X=Y the capacity is

$$C = {max}_{{p ( U \vert S )}_{X = f ( U , S )}} ( I ( U ; X ) - I ( U ; S ) ) = {max}_{{p ( U \vert S )}_{X = f ( U , S )}} ( H ( U \vert S ) - H ( U \vert X ) ) C = {max}_{{p ( U \vert S )}_{X = f ( U , S )}} ( I ( U ; X ) - I ( U ; S ) ) = {max}_{{p ( U \vert S )}_{X = f ( U , S )}} ( H ( U \vert S ) - H ( U \vert X ) ) .$$

Let U be a uniform random variable (independent of S) distributed over the set {1, . . . , K}, and define the random variable X:=f(S, U)=S(U), then H(U|S)=H(U)=log K. Furthermore, since the sets in the support of S are disjoint, then given X, one can recover the value of U, i.e., U is a function of X and thus H(U|X)=0. To conclude, by the random variable U and the function f, the channel's capacity is at least H(U|S)−H(U|X)=log K, and the result follows.

Theorem 5 shows no rate loss compared with the general Random Subsets Channel, where states are known both at the decoder and encoder. Indeed, although the exact partition of the alphabet is unknown to the decoder, it can learn it from a fixed, long enough, and known pilot (training) signal sent by the encoder at the beginning of the communication. After which, the decoder with high probability will recover the partition, and the channel will become a perfectly clean channel with an alphabet of size K. When the codeword length tends to infinity, the training signal's length is negligible, and therefore it does not incur any rate loss.

### Example 9: Completely Random Subsets Channel

In this channel model, the subsets are drawn uniformly at random from all

$$( \begin{matrix}\vert χ \vert \\ K\end{matrix} )$$

possible subsets of size K over the alphabet χ, i.e., for any s

$$⊂ χ , \vert s \vert = K , p ( S = s ) = {( \begin{matrix}\vert χ \vert \\ K\end{matrix} )}^{- 1} s ⊂ χ , \vert s \vert = K , p ( S = s ) = {( \begin{matrix}\vert χ \vert \\ K\end{matrix} )}^{- 1} .$$

This channel models a genome in which the mutations are DNA location-dependent.

Here, Theorem 4 is invoked to derive an achievable coding rate for this channel, which is a lower bound on the channel's capacity. The following theorem gives an expression for the channel capacity lower bound as a constrained optimization problem.

Theorem 6. A lower bound on the Completely Random Subsets Channel capacity is given by

$$( 13 )$$

$$C \geq {max}_{\{ {p}_{u , s} \}} \{ - \frac{1}{( \begin{matrix}\vert χ \vert \\ K\end{matrix} )} {\sum }_{u = 1}^{K} ( {\sum }_{s = 1}^{( \begin{matrix}\vert χ \vert \\ K\end{matrix} )} {p}_{u , s} log {p}_{u , s} - {\sum }_{x = 1}^{\vert χ \vert} ( {\sum }_{s : s ( u ) = x} {p}_{u , s} ) log \frac{{\sum }_{s : s ( u ) = x} {p}_{u , s}}{{\sum }_{u = 1}^{K} {\sum }_{s : s ( u ) = x} {p}_{u , s}} ) \}$$

$$s . t . {\{ {p}_{u , s} \}}_{u = 1 , s = 1}^{u = K , s = ( \begin{matrix}\vert χ \vert \\ K\end{matrix} )} \geq 0 , ∀ s ∈ \{ 1 , … , ( \begin{matrix}\vert χ \vert \\ K\end{matrix} ) \} : {\sum }_{u = 1}^{K} {p}_{u , s} = 1$$

$$C \geq {max}_{\{ {p}_{u , s} \}} \{ - \frac{1}{( \begin{matrix}\vert χ \vert \\ K\end{matrix} )} {\sum }_{u = 1}^{K} ( {\sum }_{s = 1}^{( \begin{matrix}\vert χ \vert \\ K\end{matrix} )} {p}_{u , s} log {p}_{u , s} - {\sum }_{x = 1}^{\vert χ \vert} ( {\sum }_{s : s ( u ) = x} {p}_{u , s} ) log \frac{{\sum }_{s : s ( u ) = x} {p}_{u , s}}{{\sum }_{u = I}^{K} {\sum }_{s : s ( u ) = x} {p}_{u , s}} ) \}$$

$$s . t . {\{ {p}_{u , s} \}}_{u = 1 , s = 1}^{u = K , s = ( \begin{matrix}\vert χ \vert \\ K\end{matrix} )} \geq 0 , ∀ s ∈ \{ 1 , … , ( \begin{matrix}\vert χ \vert \\ K\end{matrix} ) \} : {\sum }_{u = 1}^{K} {p}_{u , s} = 1$$

Proof. The channel capacity is given by Theorem 4. It is assumed here that the random variable U is defined over the alphabet {1, . . . , K}, in such a way that X=S(U).This assumption simplifies the following derivations and suggests that we end up with a lower bound on the capacity.

Assume some bijective mapping function between all possible sets s s containing K distinct elements from χ to the set of integers

$$\{ 1 , … , ( \begin{matrix}\vert χ \vert \\ K\end{matrix} ) \} .$$

The optimized probability distribution p(u|s), according to our assumption on U, is of the form

$$p ( u = i \vert s = j ) = {p}_{i , j} , i ∈ \{ 1 , … , K \} , j ∈ \{ 1 , … , ( \begin{matrix}\vert χ \vert \\ K\end{matrix} ) \} , {\sum }_{i = 1}^{K} {p}_{i , j} = 1 p ( u = i \vert s = j ) = {p}_{i , j} , i ∈ \{ 1 , … , K \} , j ∈ \{ 1 , … , ( \begin{matrix}\vert χ \vert \\ K\end{matrix} ) \} , {\sum }_{i = I}^{K} {p}_{i , j} = 1 \qquad ( 14 )$$

First, notice that whenever the encoder obeys the subsets constraint, X=Y, and therefore we are left with maximizing

$$\begin{matrix}{C}_{G P} = {max}_{{p ( u \vert s )}_{x = f ( u , s )}} ( I ( U ; X ) - I ( U ; S ) ) = {max}_{{p ( u \vert s )}_{x = f ( u , s )}} ( H ( U \vert S ) - H ( U \vert X ) ) \geq {max}_{{p ( u \vert s )}_{x = s ( u )}} ( H ( U \vert S ) - H ( U \vert X ) ) {C}_{G P} = {max}_{{p ( u \vert s )}_{x = f ( u , s )}} ( I ( U ; X ) - I ( U ; S ) ) = {max}_{{p ( u \vert s )}_{x = f ( u , s )}} ( H ( U \vert S ) - H ( U \vert X ) ) \geq {max}_{{p ( u \vert s )}_{x = s ( u )}} ( H ( U \vert S ) - H ( U \vert X ) ) . \\ Recall that \\ H ( U \vert S ) = - {\sum }_{s} p ( s ) H ( U \vert S = s ) = - {\sum }_{s} p ( s ) {\sum }_{u} p ( u \vert s ) log p ( u \vert s ) H ( U \vert S ) = - {\sum }_{s} p ( s ) H ( U \vert S = s ) = - {\sum }_{s} p ( s ) {\sum }_{u} p ( u \vert s ) log p ( u \vert s )\end{matrix} \qquad ( 15 )$$

$$And$$

$$H ( U \vert X ) = - {\sum }_{x} p ( x ) {\sum }_{u} p ( u \vert x ) log p ( u \vert x ) = - {\sum }_{u} {\sum }_{x} p ( u , x ) log \frac{p ( u , x )}{p ( x )} == - {\sum }_{u} {\sum }_{x} ( {\sum }_{s} p ( u , x \vert s ) p ( s ) ) log \frac{{\sum }_{s} p ( u , x \vert s ) p ( s )}{{\sum }_{u} {\sum }_{s} p ( x \vert u , s ) p ( u \vert s ) p ( s )} H ( U \vert X ) = - {\sum }_{x} p ( x ) {\sum }_{u} p ( u \vert x ) log p ( u \vert x ) = - {\sum }_{u} {\sum }_{x} p ( u , x ) log \frac{p ( u , x )}{p ( x )} == - {\sum }_{u} {\sum }_{x} ( {\sum }_{s} p ( u , x \vert s ) p ( s ) ) log \frac{{\sum }_{s} p ( u , x \vert s ) p ( s )}{{\sum }_{u} {\sum }_{s} p ( x \vert u , s ) p ( u \vert s ) p ( s )} \qquad ( 16 )$$

$$Notice that$$

$${\sum }_{s} p ( u , x \vert s ) p ( s ) = {\sum }_{s} p ( x \vert u , s ) p ( u \vert s ) p ( s ) {=}^{X = S ( U )} {\sum }_{s} \{ \begin{matrix}1 & x = s ( u ) \\ 0 & else\end{matrix} \cdot p ( u \vert s ) p ( s ) = \frac{1}{( \begin{matrix}\vert χ \vert \\ K\end{matrix} )} {\sum }_{s : s ( u ) = x} p ( u \vert s ) {\sum }_{s} p ( u , x \vert s ) p ( s ) = {\sum }_{s} p ( x \vert u , s ) p ( u \vert s ) p ( s ) {=}^{X = S ( U )} {\sum }_{s} \{ \begin{matrix}1 & x = s ( u ) \\ 0 & else\end{matrix} \cdot p ( u \vert s ) p ( s ) = \frac{1}{( \begin{matrix}\vert χ \vert \\ K\end{matrix} )} {\sum }_{s : s ( u ) = x} p ( u \vert s ) \qquad ( 17 )$$

$$And$$

$${\sum }_{u} {\sum }_{s} p ( x \vert u , s ) p ( u \vert s ) p ( s ) = \frac{1}{( \begin{matrix}\vert χ \vert \\ K\end{matrix} )} {\sum }_{u = 1}^{K} {\sum }_{s : s ( u ) = x} p ( u \vert s ) {\sum }_{u} {\sum }_{s} p ( x \vert u , s ) p ( u \vert s ) p ( s ) = \frac{1}{( \begin{matrix}\vert χ \vert \\ K\end{matrix} )} {\sum }_{u = 1}^{K} {\sum }_{s : s ( u ) = x} p ( u \vert s ) \qquad ( 18 )$$

Plugging in (17), (18) into the conditional entropy term H(U|X) (16), one gets

$$H ( U \vert X ) = - \frac{1}{( \begin{matrix}\vert χ \vert \\ K\end{matrix} )} {\sum }_{u = 1}^{K} {\sum }_{x = 1}^{\vert χ \vert} ( {\sum }_{s : s ( u ) = x} p ( u \vert s ) ) log \frac{{\sum }_{s : x = s ( u )} p ( u \vert s )}{{\sum }_{U = 1}^{K} {\sum }_{s : x = s ( u )} p ( u \vert s )} H ( U \vert X ) = - \frac{1}{( \begin{matrix}\vert χ \vert \\ K\end{matrix} )} {\sum }_{u = 1}^{K} {\sum }_{x = 1}^{\vert χ \vert} ( {\sum }_{s : s ( u ) = x} p ( u \vert s ) ) log \frac{{\sum }_{s : x = s ( u )} p ( u \vert s )}{{\sum }_{U = 1}^{K} {\sum }_{s : x = s ( u )} p ( u \vert s )} \qquad ( 19 )$$

and therefore

$$C \geq {max}_{{{\{ {p}_{u , s} \}}_{u = 1 , s = 1}^{u = K , s = ( \begin{matrix}\vert χ \vert \\ K\end{matrix} )} \geq 0}_{{\sum }_{u = 1}^{K} {p}_{u , s} = 1}} \{ H ( U \vert S ) - H ( U \vert X ) \} = {max}_{{{\{ {p}_{u , s} \}}_{u = 1 , s = 1}^{u = K , s = ( \begin{matrix}\vert χ \vert \\ K\end{matrix} )} \geq 0}_{{\sum }_{u = 1}^{K} {p}_{u , s} = 1}} \{ - \frac{1}{( \begin{matrix}\vert χ \vert \\ K\end{matrix} )} {\sum }_{u = 1}^{K} ( {\sum }_{s = 1}^{( \begin{matrix}\vert χ \vert \\ K\end{matrix} )} {p}_{u , s} log {p}_{u , s} - {\sum }_{x = 1}^{\vert χ \vert} ( {\sum }_{s : s ( u ) = x} {p}_{u , s} ) log \frac{{\sum }_{s : x = s ( u )} p ( u \vert s )}{{\sum }_{U = 1}^{K} {\sum }_{s : x = s ( u )} p ( u \vert s )} ) \} C \geq {max}_{{{\{ {p}_{u , s} \}}_{u = 1 , s = 1}^{u = K , s = ( \begin{matrix}\vert χ \vert \\ K\end{matrix} )} \geq 0}_{{\sum }_{u = 1}^{K} {p}_{u , s} = 1}} \{ H ( U \vert S ) - H ( U \vert X ) \} = {max}_{{{\{ {p}_{u , s} \}}_{u = 1 , s = 1}^{u = K , s = ( \begin{matrix}\vert χ \vert \\ K\end{matrix} )} \geq 0}_{{\sum }_{u = 1}^{K} {p}_{u , s} = 1}} \{ - \frac{1}{( \begin{matrix}\vert χ \vert \\ K\end{matrix} )} {\sum }_{u = 1}^{K} ( {\sum }_{s = 1}^{( \begin{matrix}\vert χ \vert \\ K\end{matrix} )} {p}_{u , s} log {p}_{u , s} - {\sum }_{x = 1}^{\vert χ \vert} ( {\sum }_{s : s ( u ) = x} {p}_{u , s} ) log \frac{{\sum }_{s : x = s ( u )} p ( u \vert s )}{{\sum }_{U = 1}^{K} {\sum }_{s : x = s ( u )} p ( u \vert s )} ) \} .$$

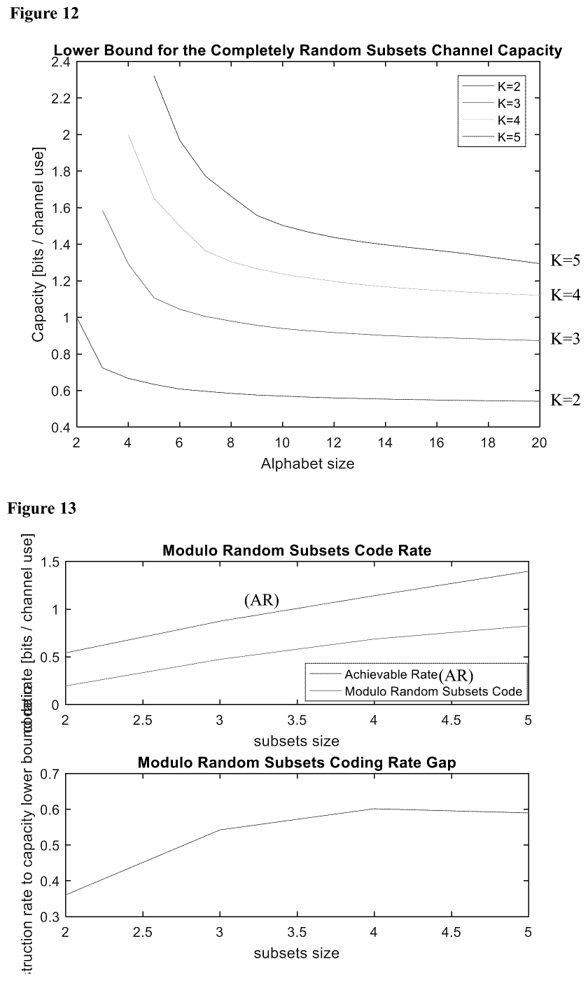

As mentioned, Theorem 6 gives an expression for an achievable coding rate (capacity lower bound) for the Completely Random Subsets Channel capacity as a closed-form constrained optimization problem. When the optimal random variable U in Theorem 4 indeed holds our assumptions (defined over the alphabet {1, . . . , K}, in such a way that X=S(U)), the lower bound is tight, and Theorem 6 gives the channel capacity. The lower bound in Theorem 6 can be solved numerically, as shown in FIG. 12.

Notice that each graph starts at the point K=[χ], where the channel capacity is log K as expected since it is a perfectly clean channel. The capacity lower bound decreases as the alphabet size increases and K is fixed due to the growing number of K-subsets.

Although Theorem 6 and FIG. 12 shed some light on the achievable coding rate (or maybe even on the channel's capacity), one needs to find explicit code constructions, to be able to transmit information at a rate close to the capacity. Hereinbelow, two code constructions for the Random Subsets Channel are provided. But first, two examples of simple coding strategies are examined that will provide some intuition for future constructions. Also, the first coding strategy is causal, whereas the second utilizes the non-causality of the states to achieve a large code rate.

Example 9i. Assume that K=3, χ={1,2,3,4}. Any subset S of size 3 satisfies min(S)∈{1,2} and max(S)∈{3,4}, then given a bit b to be transmitted, the encoder sets x=min(S)∈{1,2} if b=0, otherwise, it sets x=max(S)∈{3,4}. It is clear that upon receiving x, the decoder can recover the value of b, as needed.

This coding strategy is causal, as the current bit encoding does not depend on future channel states. Compared to the channel capacity lower bound C=1.293, the scheme's rate is R=1 bits per channel usage.

Example 9ii. Assume that K=2, χ={1,2,3}. Given two consecutive channel states S₁, S₂ and a bit b to be transmitted, the encoder sets x₁, x₂ according to Table 1. For example, assume that S₁=(1,2), S₂=(1,3) then to encode the bit 0, the encoder sends the vector (1,1); otherwise, it sends the vector (2,3). Notice that there is no ambiguity in the encoded symbols x₁, x₂ (the two right columns don't share a common pair of encoded symbols). Therefore, the decoder can recover the transmitted bit. Compared to the channel capacity lower bound C=0.723, the scheme's rate is R=0.5 bits per channel usage.

The coding scheme is non-causal, as the encoding of the current symbol depends on the current and the following channel states. Notice that there is no coding scheme that encodes one bit of information using one channel state (as expected, since the channel capacity in this case is C=0.723<1). (See Table 1)

Table 1: Random Subsets Channel: simple non-causal coding strategy for an alphabet of 3 symbols and a subset size of 2 symbols.

| Two consecutive | Encoding for | Encoding for |
|---|---|---|
| channel subsets | information bit ‘0.’ | information bit ‘1.’ |
| (1, 2) (1, 2) | 1, 1 | 2, 2 |
| (1, 2) (1, 3) | 1, 1 | 2, 3 |
| (1, 2) (2, 3) | 1, 2 | 2, 2 |
| (1, 3) (1, 2) | 1, 1 | 3, 1 |
| (1, 3) (1, 3) | 1, 1 | 3, 1 |
| (1, 3) (2, 3) | 1, 2 | 3, 3 |
| (2, 3) (1, 2) | 2, 1 | 3, 1 |
| (2, 3) (1, 3) | 2, 1 | 3, 1 |
| (2, 3) (2, 3) | 3, 2 | 3, 3 |

### Example 10: Code Constructions for the Random Subsets Channel

Herein is provided code constructions for the Random Subsets Channel, independent of the subset's probability distribution p(s), and therefore applicable to both Partial and Completely Random Subsets Channels. However, the focus is on the Completely Random Subsets model.

Modulo Random Subsets Code: The key idea of the construction relies on the following. If the encoder is always guaranteed that each state contains both even and odd integers, it could transmit at a rate of one bit by using the bit's parity. However, such an assumption does not hold in the model; hence the idea is to force some structure.

Encoding. Let q be an integer that divides x and let C be a code of length n and rate

$$R = {C}_{qSC} {log}_{q} 2 , \qquad ( 20 )$$

over the alphabet {0, . . . , q−1}that achieves the capacity of the q-ary symmetric channel

$${C}_{qSC} = log q - {h}_{2} ( {p}_{f} ) - {p}_{f} log ( q - 1 ) = log q + {p}_{f} log {p}_{f} + ( 1 - {p}_{f} ) log ( 1 - {p}_{f} ) - {p}_{f} log ( q - 1 ) , \qquad ( 21 )$$

Where the symbol error probability p_{f} is to be determined. Let c=c₁, . . . , cₙ∈C be the encoding of the information to be transmitted. The iₜₕ symbol xᵢ to be transmitted over the channel is set to be s∈Sᵢ if s mod q=cᵢ. Otherwise, if there is no such symbol, it is set to be an arbitrary element of Sᵢ.The resulting transmitted codeword is x=x₁, . . . , xₙ, which never violates the subsets constraint.

Decoding. Since the codeword x does not violate the subset constraints, the decoding of the received codeword y=x∈χⁿ is straightforward. The receiver first computes the vector (x₁ mod q , . . . , xₙ mod q), which is then passed through a decoder of the code C.

Code rate analysis. The received vector contains an error at the iₜₕ coordinate, i.e., xᵢ mod q≠cᵢ, if and only if there is no symbol s∈Sᵢ such s mod q=cᵢ. The probability of this event is easily calculated. Indeed, since for each 0≤i≤q−1 there are exactly |χ|/q integers, s∈{0, . . . , |χ|−1}, s mod q=i then the required probability of error is

$${p}_{f} = \frac{( \begin{matrix}\vert χ \vert ( q - 1 ) / q \\ K\end{matrix} )}{( \begin{matrix}\vert χ \vert \\ K\end{matrix} )} , \qquad ( 22 )$$

It is clear by symmetry that given that an error has occurred, the probability of any of the q−1 other symbols is equal. To conclude, the channel boils down to a q-SC with probability of error p_{f} as above. By choosing the code C to be capacity-achieving, it is clear that for large values of n the decoding error tends to zero, and the overall rate (number of bits per channel usage) is equal to C_{qSC} with parameter p_{f}. Plugging (22), (21) into (20) results in the code rate of the construction.

The integer q≤ K is a free parameter of the code and should be chosen to maximize the code rate R. The following figure shows the optimal coding rate as a function of the length of the subset K, compared to the completely random channel achievable coding rate derived earlier in this section when |χ|=64, K∈{2,3,4,5}. Also, the coding rate gap, defined by the ratio of the construction code rate to the derived channel capacity lower bound (Theorem 6), is depicted. For all numerical evaluations, q=2 was the optimal choice (FIG. 13).

Linear Random subsets code: This section presents another code construction for the Completely Random Subsets Channel with a higher rate than the previous construction. However, it can be achieved at the expense of higher complexity.

Let the mapping Cᵢₙ: v^{q}→χⁿ be the inner code, Cₒᵤₜ: (v^{q})^{Q}→(χ^{q})^{N} be the outer code, and a symbol of χ^{q} is referred to as a super-symbol. Furthermore, we assume that |χ| is a power of a prime, and therefore there exists a finite field of order |χ|.

Encoding. An information vector ν∈χ^{q·Q} to be transmitted is encoded into a vector ν′∈χ^{n·N} as follows. Using the outer code Cₒᵤₜ, encode ν (viewed as a vector of Q super-symbols) to a vector w∈(χ^{q})^{N}. Using the inner code Cᵢₙ, encode each of the N super-symbols of w, to a vector of length n over χ. The final codeword ν′ is of length n·N over χ. The outer code Cₒᵤₜ is chosen to be an [N, Q] code over the alphabet χ^{q}, with rate

$${R}_{outer} = \frac{Q}{N} .$$

Now is described the encoding of the inner encoding step. To be more mathematically precise, the inner code Cᵢₙ is a function of the input vector and the states vector. Hence, each information vector can be encoded to several different codewords, and the precise codeword is chosen according to the state vector. The formal definition is as follows. Let H be a predetermined fixed matrix of order q×n over χ and rank(H)=q known both to the encoder and decoder. It is explained hereinbelow how H is chosen. Given a vector u∈χ^{q} to be encoded and a state vector s=S₁ . . . Sₙ, the inner encoding of u is a vector u′∈χⁿ that satisfies

$$∀ i ∈ \{ 1 , … , n \} : {u}_{i}^{′} ∈ {S}_{i} , u = H \cdot {u}^{′} \qquad ( 23 )$$

If there are several such vectors u′, then the encoder picks one of them arbitrarily. If there are no such vectors, then the encoder arbitrarily sets u′ to be one of the vectors from the set s=S₁ . . . Sₙ.

Decoding. The decoder first applies the inner decoder for each of the N channel's output vectors u′∈χⁿ and decodes it into the super-symbol u∈χ^{q} using (23), which amounts to simple matrix multiplication. Note that the decoder does not need to know the channel states (subsets) s=S₁. . . Sₙ. Then, it applies the outer decoder to the N super-symbols outputted by the inner decoder to recover the q·Q information symbols.

Code analysis. Errors can occur only at the inner codes' encoding step, whenever there is no solution to (23). In such a case, the inner code randomly picks one of the vectors that satisfy the subset constraints. Since the states are uniformly distributed among all possible subsets, then each error is uniformly distributed among all |χ|^{q}−1 possible super-symbols. Hence, as in the previous construction, the channel boils down to a qSC defined over the alphabet χ^{q} and error probability p_{f}. Similarly, the outer code is chosen to have a coding rate equal to the capacity of a qSC over the alphabet χ^{q} with parameter p_{f},

$${R}_{outer} = {C}_{qSC} = log \vert {χ}^{q} \vert - {h}_{2} ( {p}_{f} ) - {p}_{f} log ( \vert {χ}^{q} \vert - 1 ) = log \vert {χ}^{q} \vert + {p}_{f} log {p}_{f} + ( 1 - {p}_{f} ) log ( 1 - {p}_{f} ) - {p}_{f} log ( \vert {χ}^{q} \vert - 1 ) , \qquad ( 24 )$$

So that the outer decoder can correct all super-symbol errors that occurred by the inner encoder and perfectly reconstruct the original information message. Transmission of one super-symbol involves the transmission of q information symbols by n channel uses of the original Completely Random Subsets Channel, and finally, the overall coding rate of the above construction is

$$R = \frac{1}{n} {R}_{outer} = \frac{1}{n} ( log \vert {χ}^{q} \vert + {p}_{f} log {p}_{f} + ( 1 - {p}_{f} ) log ( 1 - {p}_{f} ) - {p}_{f} log ( \vert {χ}^{q} \vert - 1 ) ) \qquad ( 25 )$$

bits per channel use.

To derive the coding rate of this code construction, a numerical simulation was performed that evaluates the super-symbol error probability p_{f}.

Numerical simulation: The following Monte Carlo simulation was performed for |χ|=64. First, independently and uniformly at random each entry of the matrix H from the finite field GF(|χ|=2⁶=64) was picked, such that rank(H)=q. Next, the information vector u to be an iid uniform random vector of q elements over GF(|χ|) was picked. Finally, n subsets (states) of size K of χ were randomly picked independently and uniformly and it was verified that (23) holds. The random selection of the subsets was repeated for a fixed H to estimate the error probability p_{f}, and calculated the code rate using (25). Such a simulation numerically finds the code rate for a specific H. The simulation was repeated for random selections of H, and the resulting code rates of all evaluations were averaged to finally get a numerical value for the construction's code rate.

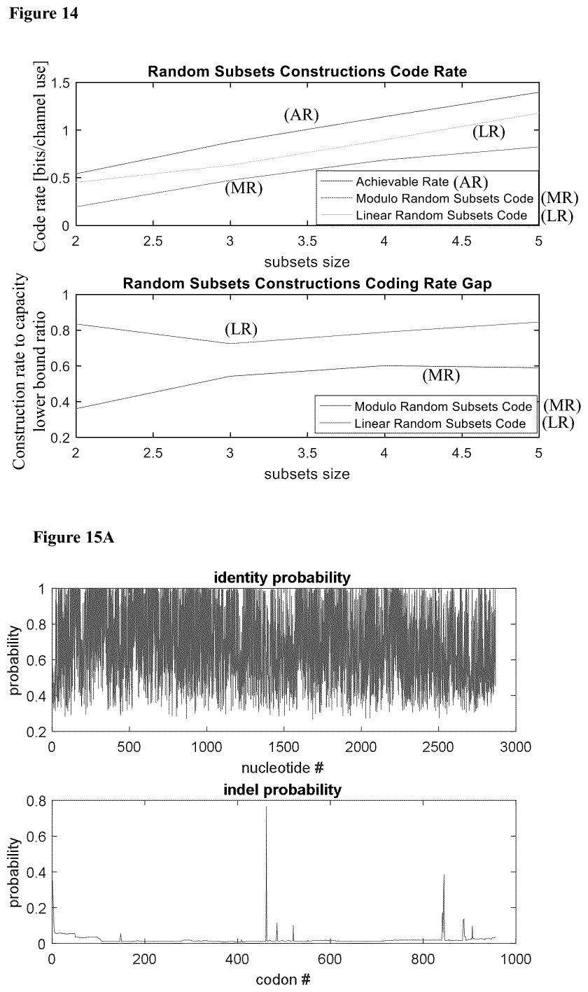

FIG. 14 depicts the simulation result for |χ|=64 and variable subsets length K. In this figure, one can see that the Linear Random Subsets Code construction performs better than the Modulo Random Subsets construction from the previous section, in cost of encoding complexity. In the Linear Random Subsets Code construction, we need to go over Kⁿ possible vectors over χⁿ and look if (23) holds in order to transmit q information symbols, where in the Modulo Random Subsets construction, we only performed simple modulo calculation of q elements.

### Example 11: Error Probabilities

The following simple assumptions are made to calculate the error probabilities Pₛ,Pₑ. It is assumed that all orthologs are time distant from each other by N_{G} generations. That means that the number of generations passed between each pair of orthologs is N_{G}. Such an assumption simplifies the statistical measures calculations. Also, it is assumed that interested lies in the error probabilities after M_{G} generations, which is the number of generations passed along the storage time (time elapsed between encoding and decoding). Finally, it is assumed that each generation may cause a nucleotide substitution with constant probability, independent of past generations.

Following these assumptions, after performing the DNA alignment, the identity probabilities P_{id}(k), k∈S, which are the identity probabilities of all nucleotides in the site of interest S (the site in which the code will operate), remain unchanged over N_{G} generations. Averaging all identity probabilities over all nucleotides in the site of interest S, one gets

$${P}_{i d} = \frac{1}{\vert s \vert} {\sum }_{k ∈ S} {P}_{i d} ( k ) .$$

According to this model assumptions, P_{id} should equal the number of even number of substitutions after N_{G} generations. Denote by X the number of substitutions after N_{G} generations. X is the sum of N_{G} iid Bernoulli trials with parameter p, where p is the probability of substitution in a single generation. The probability of an even or odd value of X is

$$Pr ( Xiseven ) + Pr ( Xisodd ) = {( ( 1 - p ) + p )}^{{N}_{G}} = {\sum }_{k = 0}^{{N}_{G}} ( \begin{matrix}{N}_{G} \\ k\end{matrix} ) {{p}^{k} ( 1 - p )}^{{N}_{G} - k} = = {{\sum }_{k = 0}}^{⌊ \frac{{N}_{G}}{2} ⌋} ( \begin{matrix}{N}_{G} \\ 2 k\end{matrix} ) {{p}^{2 k} ( 1 - p )}^{{N}_{G} - 2 k} + {{\sum }_{k = 0}}^{⌊ \frac{{N}_{G}}{2} ⌋} ( \begin{matrix}{N}_{G} \\ 2 k + 1\end{matrix} ) {{p}^{2 k + 1} ( 1 - p )}^{{N}_{G} - 2 k - 1}$$

$$Pr ( Xiseven ) - Pr ( Xisodd ) = {( ( 1 - p ) - p )}^{{N}_{G}} = {\sum }_{k = 0}^{{N}_{G}} ( \begin{matrix}{N}_{G} \\ k\end{matrix} ) {( - p )}^{k} {( 1 - p )}^{{N}_{G} - k} == {{\sum }_{k = 0}}^{⌊ \frac{{N}_{G}}{2} ⌋} ( \begin{matrix}{N}_{G} \\ 2 k\end{matrix} ) {{p}^{2 k} ( 1 - p )}^{{N}_{G} - 2 k} - {{\sum }_{k = 0}}^{⌊ \frac{{N}_{G}}{2} ⌋} ( \begin{matrix}{N}_{G} \\ 2 k + 1\end{matrix} ) {{p}^{2 k + 1} ( 1 - p )}^{{N}_{G} - 2 k - 1} ⇒ \{ \begin{matrix}P r ( X i s e v e n ) = \frac{1}{2} ( 1 + {( 1 - 2 p )}^{{N}_{G}} ) \\ P r ( X i s o d d ) = \frac{1}{2} ( 1 - {( 1 - 2 p )}^{{N}_{G}} )\end{matrix}$$

Identity is equivalent to an even value of X. Therefore one can write ½(1+(1−2p)^{N}^{_{G}})=P_{id}⇒p=½(1−(2P_{id}−1)^{1/N}^{_{G}}). Finally, we are interested in the probability of substitution event after M_{G} generations, which equals to an odd number of successes in M_{G} iid Bernoulli trials with parameter p, or Pₛ=½(1−(1−2p)^{M}^{_{G}})=½(1−(2P_{id}−1)^{M}^{_{G}}^{/N}^{_{G}}). Recall that one wants the average identity probability so that

$${P}_{s} = \frac{1}{2} ( 1 - {( 2 \frac{1}{\vert S \vert} {\sum }_{k ∈ S} {P}_{i d} ( k ) - 1 )}^{{M}_{G} / {N}_{G}} ) \qquad ( 26 )$$

(26) gives the substitution probability for DNA storage along with M_{G} generations. An average identity probability of P_{id} is assumed, calculated over all nucleotides in the site of interest and is based on alignment of orthologs all separated by N_{G} generations.

An erasure event is assumed to occur at the whole site level, meaning the entire site S is erased or not. As mentioned, the actual biological phenomena causing an erasure can be an actual extinction of a species. Still, it can also be an indel, in which only a short sequence of nucleotides may be deleted.

The following simple estimate for an erasure probability is used. Given that all indel probabilities estimated from the alignment, P_{indel}(k), k∈S, are correlative along with all nucleotides in the site of interest S (as defined in the sequel), the maximal indel probability of one of the nucleotides in the site is the erasure probability,

$${P}_{e} = {max}_{k ∈ S} {P}_{indel} ( k ) \qquad ( 27 )$$

By correlation in the indel probability along the whole site, we mean a small variance of the indel estimations over the entire site of interest. The reason behind such a definition is that all the codons in the site have about the same probability of suffering from an indel, therefore an indel will be likely to occur among the whole site, an event similar to an erasure of the site.

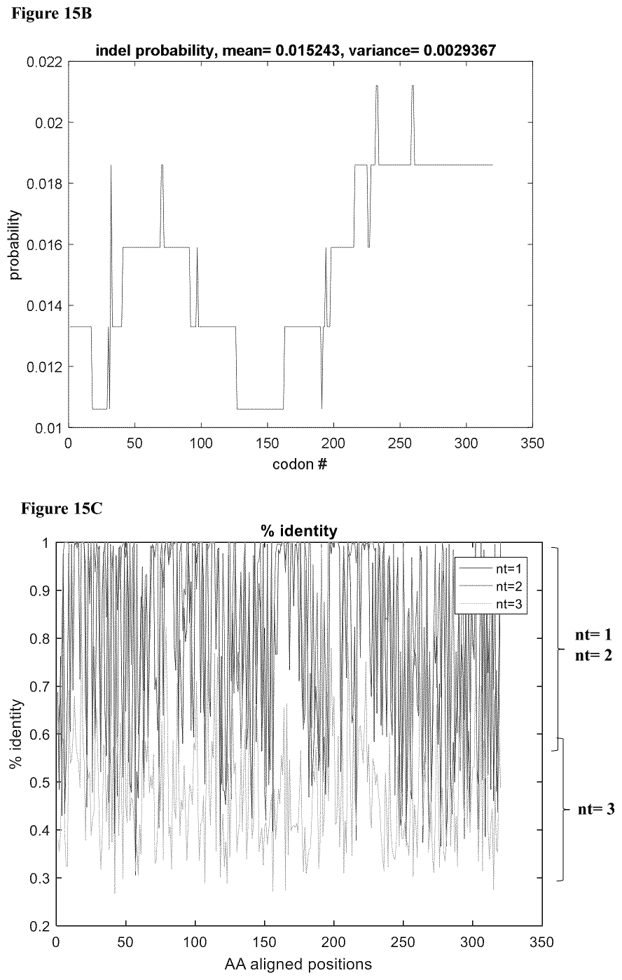

Focusing on the gene ALA1 in S. Cerevisiae as a proof of principle. The estimated identity and indel probabilities along the whole gene are given in FIG. 15A. The site of interest S is defined to include codons number 521-841, or nucleotides 1561-2521. First, one can see a correlative indel probability along that site, with a maximal value of

$${P}_{e} = {max}_{k ∈ S} {P}_{indel} ( k ) = 0.0212$$

### (FIG. 15B)

It is clear that an indel is not very likely to occur (as all probabilities are small), but if it does, then the whole site is expected to suffer it, as all nucleotides seem to be correlative.

Next, the average identity probability along all the site's nucleotides is

$${P}_{i d} = \frac{1}{\vert s \vert} {\sum }_{k ∈ S} {P}_{i d} ( k ) = 0.698 .$$

As depicted in the FIG. 15C, each third nucleotide is more likely to substitute, as most codons obey the Amino Acid dictionary constraint. Codons may suffer from synonymous changes only. In most cases, only the third nucleotide may substitute.

One generation of S. Cerevisiae lasts for approximately 90 minutes. If it is assumed that all orthologs in the alignment are separated by 1 million years or N_{G}=5.84·10⁹ generations and the storage time is 1 thousand years or M_{G}=5.84·10⁶ generations, then the substitution probability according to the model is

$${P}_{s} = \frac{1}{2} ( 1 - {( 2 \frac{1}{\vert S \vert} {\sum }_{k ∈ S} {P}_{id} ( k ) - 1 )}^{{M}_{G} / {N}_{G}} ) = 4.62 \cdot {10}^{- 4} .$$

The allowed codons changes dictionary D is defined as a mapping between each codon's location in the site of interest to a subset of codons. This subset imposes the constraint in which one can change the original codon into a member of this subset only; otherwise, the whole site will be erased (microorganism death).

The following estimation for D is suggested. The codons distribution P_{c}(k), k∈S is used, where for each DNA location k, P_{c}(k) there is a vector of 64 coordinates, estimated from the DNA alignment as the distribution function of all 64 possible codons in the current DNA location. Then, the current DNA location k∈S is mapped into a subset of codons, including only codons with associated probability value higher than a threshold T. Therefore, D is defined as the mapping function between each DNA location to a subset of codons D(k) given by

$$D : S \to D ( k ) = \{ c ∈ {\{ A , C , G , T \}}^{3} : {P}_{c} ( k ) \geq T \} \qquad ( 28 )$$

This way, the set of allowed changes in each DNA location is chosen such that each member of this set appears in at least T percent of all aligned orthologs, and a change cause by the storage is justified in the sense that evolution also performed such a change in at least T percent of the orthologs.

An interesting measure is the average subset size mapped by D along the whole site of interest S, given by

$$\frac{1}{\vert S \vert} {\sum }_{k ∈ S} \vert D ( k ) \vert \qquad ( 29 )$$

FIG. 16 compares the size of each subset of allowed changes |D(k)| along the site of interest (codons number 521-841 in ALA1) as a function of T in (28). Also, the case in which the criteria for building D is based on the AA preservation (only synonymous changes are allowed) is depicted. A higher threshold is more conservative, as it allows fewer changes, and one can see in FIG. 16 that, indeed, the average subset size is smaller.

Other definitions for the dictionary D may be considered, involving other biological aspects such as information encoded in the mRNA, affecting gene expression and fitness. A fusion between the method suggested above (based on DNA alignment) and Amino Acid preservation is introduced herein.

### Example 12: Coding Scheme

There is now provided a more detailed explanation of the scheme presented in FIG. 6.

Encoder: The encoder scheme is summarized in FIG. 17. The Encoder input is a chunk of k information bits, and the outputs are M message blocks of length L bits each. Each message block will be encoded later into a synthesized site.

The encoder generates M identifiers, which is the set {0, . . . , M−1}. Each identifier is a binary vector of length ┌log M┐ bits and is encoded solely into an encoded identifier of length L_{ID} bits using Reed-Solomon (RS) code with message length ┌log M┐ and codeword length L_{ID}, all in bits-RS(L_{id}, ┌log M┐). The actual symbol length for that RS code is an integer m_{ID} such that

$$\frac{⌈ log M ⌉}{{m}_{ID}} , \frac{{L}_{ID}}{{m}_{ID}} ∈ N , {m}_{ID} \cdot {2}^{{m}_{ID}} \geq {L}_{ID} .$$

The information bits are encoded using another RS code with message length k and codeword length M(L−L_{ID}), all in bits-RS(M(L−L_{id}), k). The actual symbol length for that RS code is an integer m such that

$$\frac{k}{m} , \frac{M ( L - {L}_{ID} )}{m} ∈ N , m \cdot {2}^{m} \geq M ( L - {L}_{ID} ) .$$

Finally, the information RS codeword is divided into M blocks, each block contains L−L_{ID} bits, and an appropriate encoded identifier is attached at the begging of each block to finally create M message blocks of length L bits each. We note that the identifiers' role is to handle the channel permutation, and the RS codes' role is to address both erasures and substitutions, as we explain herein. The total coding rate of the encoder

$${r}_{encoder} = \frac{k}{M L} .$$

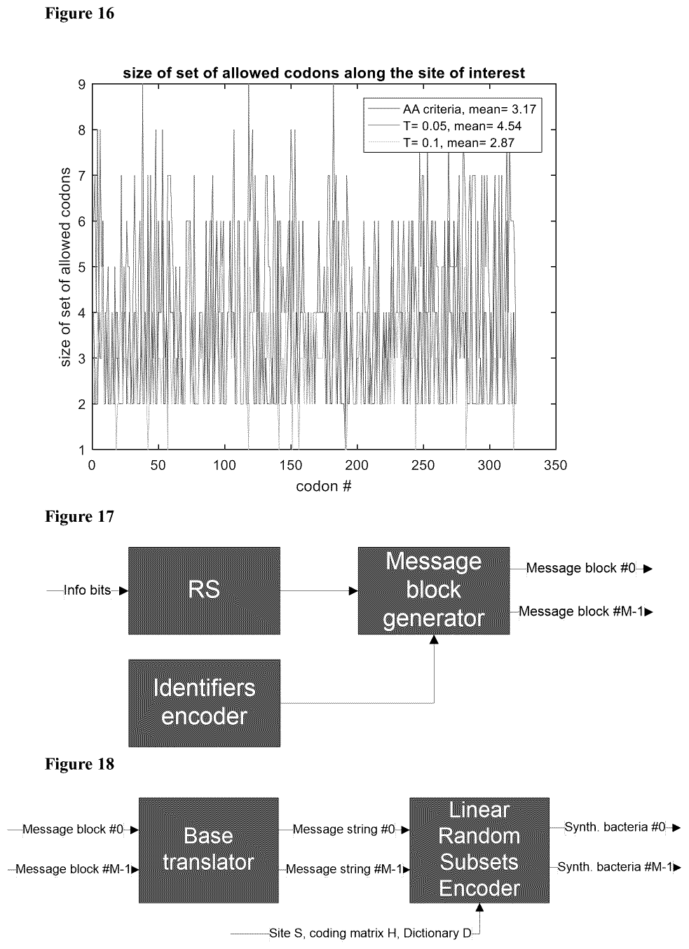

Modulator: The modulator scheme is summarized in FIG. 18. The Modulator inputs are M message blocks of length L bits each, and outputs are M synthesized sites, which are vectors of nucleotides.

The modulator's goal is to generate the synthesized sites in a way that will store the information, will not violate the allowed codons changes dictionary constraints (abbreviated as D-constraints), and will optimize the Codon Adaptation Index (CAI) measure.

As depicted in Error! Reference source not found., the modulator first translates each pair of bits into a single nucleotide using the mapping (00,01,10,11)↔(A, C, G,T). Then it takes each vector of nucleotides, which is L/2 nucleotides long, and generates a synthesized site, using the Linear Random Subsets Encoder, defined hereinabove.

The modulator holds the original site S, and the allowed codons changes dictionary D. The dictionary is either given to it or estimated using DNA alignment as discussed above. Based on the dictionary D, the modulator calculates the mean subset length along the whole site S as in (29), and finally rounding the result to evaluate an estimate for K, which is the subset size. Together with the codons alphabet χ, with |χ|=64, and the assumption of Completely Random Subsets Channel, the modulator determines the channel coding achievable rate according to the empirical value of the Linear Random Subsets Code (LRSC) rate in Error! Reference source not found.

The actual LRSC setting here is as follows. First, the outer block code is the information Reed-Solomon code, RS(M(L−L_{ID}), k), presented earlier as a part of the encoder. From now on, only the inner code is discussed.

The alphabet used in the inner code is the codon's alphabet χ, with |χ|=64. Instead of performing operations over X, the Galois field which the code will operate over is the nucleotide field, which under the mapping (A, C, G, T)↔(0, 1, α, β) is equivalent to GF(4) with the operations as in the following Table 2.

TABLE 2

| | | | | | | | |
|---|---|---|---|---|---|---|---|
| + | 0 | 1 | αβ | • | 0 | 1 | αβ |
| 0 | 0 | 1 | αβ | 0 | 0 | 0 | 00 |
| 1 | 1 | 0 | βα | 1 | 0 | 1 | αβ |
| α | α | β | 01 | α | 0 | α | β1 |
| β | β | α | 10 | β | 0 | β | 1α |

Each codon is equivalent to three symbols from GF(4), so that the D-constraints (given over χ) can be translated to GF (4). From now on, only the nucleotide field GF (4) is related to.

Next, the modulator chooses the integers q_{LRSC}, n_{LRSC} (q, n) and the inner coding matrix H, which define the inner code .q_{LRSC}, n_{LRSC} are chosen such that the coding rate following (24) does not exceed the achievable channel rate found empirically as described above, and such that the value of Kⁿ^{_{LRSC}}^{/3} is realizable in terms of computing complexity (as inner encoding will demand going over Kⁿ^{_{LRSC}}^{/3} vectors and check whether (23) holds). The inner coding matrix H is a q_{LRSC}×n_{LRSC} matrix with elements in GF (4)={A, C, G, T}.

The modulator informs the receiver with the actual H it uses for encoding. Aside from H, the receiver has no other side information.

The resulting site length, or actual number of nucleotides used to modulate the information, is

$$\vert S \vert = \frac{L \cdot {n}_{LRSC}}{2 \cdot {q}_{LRSC}} .$$

The modulating process is the same for all synthesized sites (microorganisms), so from now on, only one of them is focused upon.

As explained in hereinabove, the modulator (Linear Random Subset Encoder) will take q_{LRSC} nucleotides (2·q_{LRSC} bits) and will encode them into a subsite, which we define as a sequence of n_{LRSC} nucleotides. Here, to handle both the D-constraints and CAI optimization, the following algorithm is employed. The original site S is divided into subsites S(i), i∈

$$\{ 1 , … , \frac{L}{2 \cdot {q}_{LRSC}} \} ;$$

each one is n_{LRSC} nucleotides (or

$$\frac{{n}_{LRSC}}{3}$$

codons) long, such that

$$S = S ( 1 ) … S ( \frac{L}{2 \cdot {q}_{LRSC}} ) ∈ {\{ A , C , G , T \}}^{\vert S \vert} .$$

Also, the message string m to be stored over the current synthesized site is divided into

$$\frac{L}{2 \cdot {q}_{LRSC}}$$

sub-messages m(i), i∈

$$\{ 1 , … , \frac{L}{2 \cdot {q}_{LRSC}} \} ,$$

each one is q_{LRSC} nucleotides long, such that

$$m = m ( 1 ) … m ( \frac{L}{2 \cdot {q}_{LRSC}} ) ∈ {\{ A , C , G , T \}}^{L / 2} .$$

Each sub-message will be encoded into each sub-site according to the procedure described next.

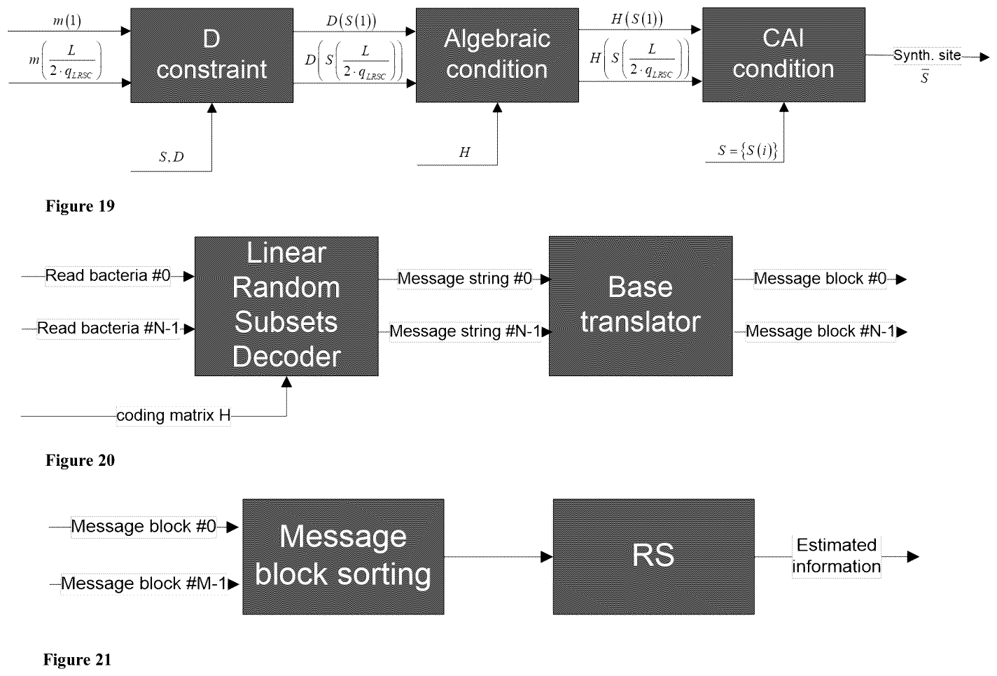

The modulator scheme for one synthesized site is depicted schematically in FIG. 19.

The iₜₕ sub-message m(i) will be encoded in its corresponding subsite S(i) in the following way. First, all D-constraints equivalents of the sub-site S(i), denoted by the set of vectors in {A, C, G, T}ⁿ^{_{LRSC}}, D(S(i)), are generated. In other words, D(S(i)) is a set of nucleotide vectors, each vector of length n_{LRSC} nucleotides, where each vector is not violating the D-constraints with respect to S(i). Then, only the vectors u′∈{A, C, G, T}ⁿ^{_{LRSC}} which realize

$$\{ \begin{matrix}H \cdot {u}^{′} = m ( i ) \\ {u}^{′} ∈ D ( S ( i ) )\end{matrix} \qquad ( 30 )$$

are kept, where His the LRSC inner matrix, and gathered into the set of nucleotide vectors H(S(i)). The set H(S(i)) of vectors in {A, C, G, T}ⁿ^{_{LRSC}} contains only subsites which hold both D-constraints and the algebraic equation defined by (30), and therefore is a valid encoding following the Linear Random Subsets Coding method.

Instead of picking one of the possible solutions (members of H(S(i))) in a random manner, the following optimality criteria are proposed.

A single vector from H(S(i)) with the smallest CAI change with respect to the original subsite is chosen. From a mathematical perspective, CAI preservation is defined as follows. First, codon's frequencies {f_{c}}_{c=0}⁶³ are calculated as the number of repetitions of that codon c over the whole genome, and then a weight metric for each codon c is calculated as

$${w}_{c} = \frac{{f}_{c}}{{max}_{p ∈ {AA}_{c}} {f}_{P}} , c = 0 , … , 63 \qquad ( 31 )$$

AA_{c} is the set of all synonymous codons with respect to c. The CAI of some sub-site vector u′∈{A, C, G, T}ⁿ^{_{LRSC}} is defined as the geometric mean of the weight metrics of all codons in the subsite,

$${CAI}_{{u}^{′}} = {( {Π}_{p = 0}^{{n}_{LRSC} / 3 - 1} {w}_{{u}^{′} ( 3 p , 3 p + 1 , 3 p + 2 )} )}^{\frac{3}{{n}_{LRSC}}} \qquad ( 32 )$$

Finally, the optimality criteria from which a single subsite from H(S(i)) is chosen is

$$\\ \begin{matrix}\overline{S} ( i ) = {arg min}_{{u}^{{}_{} ′} ∈ H ( S ( i ) )} \vert {CAI}_{S ( i )} - {CAI}_{{u}^{{}_{} ′}} \vert & ( 33 )\end{matrix}$$

It is noted that this is a greedy approach to the CAI preservation problem of the whole site S. One needs to solve

$$\\ \overline{S} = {arg min}_{\tilde{S}} \vert {CAI}_{S} - {CAI}_{\tilde{S}} \vert$$

all the subsites together a computationally comprehensive problem to solve.

Finally, the synthesized site S̅ that was chosen for the storage of the message block m, is the concatenation of all synthesized sub-sites chosen as explained (splitting the original site into sub-sites and finding a synthesized sub-site holding the D-constraints, algebraic constraint, and CAI criteria for its relevant sub-message).

Demodulator: The demodulator scheme is summarized in FIG. 20. The Demodulator inputs are N read sites, and outputs are N message blocks of length L bits each.

As seen in Error! Reference source not found, the demodulator first uses an LRSC decoder. The LRSC Decoder accepts N≤M read sites and extracts L/2 nucleotides it believes that are coded over each one of the sites, according to the shared LRSC inner matrix H and the algebraic structure it dictates. Namely, for some read site

$$\\ S = S ( 1 ) … S ( \frac{L}{2 \cdot {q}_{LRSC}} ) ∈ {\{ A , C , G , T \}}^{\vert S \vert} ,$$

the LRSC Decoder performs

$$\\ ∀ i ∈ \{ 1 , … , \frac{L}{2 \cdot {q}_{LRSC}} \} : m ( i ) = H \cdot S ( i ) ,$$

where S(i)∈{A, C, G, T}ⁿ^{_{LRSC}} are the read sub-sites and m(i)∈{A, C, G, T}^{q}^{_{LRSC}} are the extracted sub-messages. The decoded message string for some read site is

$$\\ m = m ( 1 ) … m ( \frac{L}{2 \cdot {q}_{LRSC}} ) ∈ {\{ A , C , G , T \}}^{L / 2} ,$$

and finally, each message string of L/2 nucleotides is translated into L bits to form the demodulated message blocks.

Decoder: The decoder scheme is summarized in FIG. 21. The Decoder inputs are N erroneous message blocks, and output is the estimated original information chunk of k information bits.

The decoder first sorts the messages according to their original order using the encoded identifiers. Each encoded identifier is read from the first L_{ID} bits of each message block and corrected using its protecting RS code and additional information of the expected set of all numbers in the set {0, . . . , M−1}, using an optimal Maximum Likelihood approach. That is, instead of a regular RS decoder operating over each identifier, we decode all identifiers together using a Maximum Likelihood algorithm which allocates each expected identifier in the set {0, . . . , M−1} with the most likely channel output identifier.

Once the messages are sorted, the identifiers are removed from each message block, and all message blocks are concatenated in the order recovered by the identifiers. Then, an RS decoder with message length k and block length M(L−L_{ID}) is used to correct both message block erasures (correspond to a burst of RS symbols erasures) and nucleotides substitutions (correspond to RS symbol substitution).

### Example 13: Encoder-Decoder Performance Evaluation

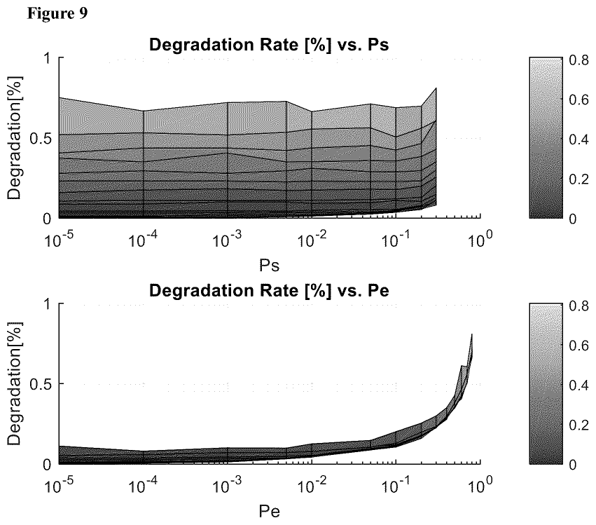

To test the coding scheme performance, a simulation was performed comparing the coding scheme of the invention's performance to the bounds derived in FIGS. 7-9. The simulation includes only the encoder, channel, and decoder, excluding the modulator and demodulator. The channel simulated was the Noisy Shuffling Sampling Channel.

One may recall that the higher bound on the coding rate is the classical capacity. The lower bound is a lower bound on achievability for coding under the Finite Block length regime.

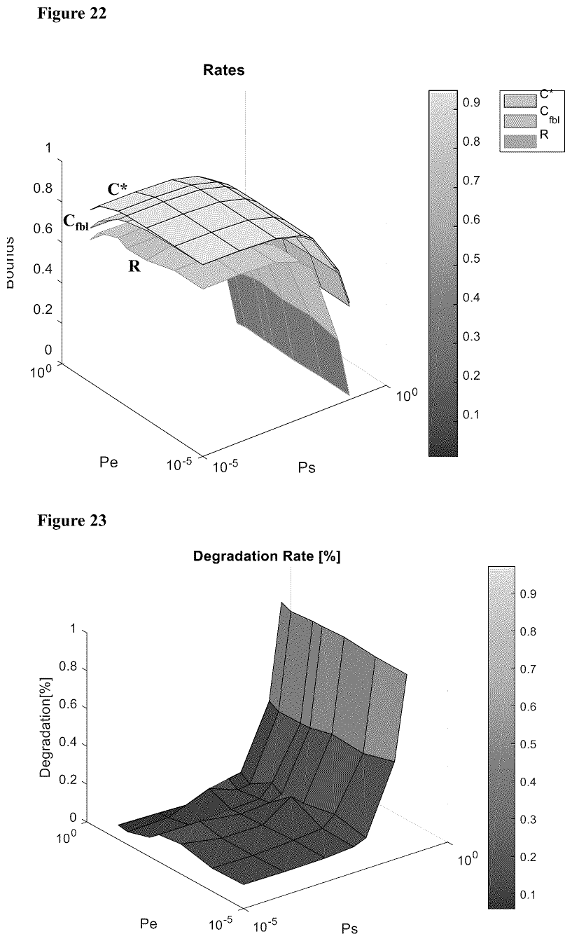

The simulation fixed parameters are M=180, L=144, ε=10⁻⁶, and the varying parameters are Pₑ, Pₛ. The performance metric is the code rate, defined by the ratio between the number of information bits k to the total codeword length m=M·L. It is optimized to the highest possible value resulting in no decoding errors. Here, CFBL defines the finite block-length lower bound and by C^{*} the channel capacity in “DNA-Based Storage: Models and Fundamental Limits.”, Ilan Shomorony and Reinhard Heckel, IEEE transactions on information theory, vol. 67, no. 6, June 2021, herein incorporated by reference in its entirety (or (1)). The result is shown in FIG. 22.

Next, the normalized degradation rate was simulated, which is defined as the relative gap between the schema coding rate and lower bound,

$$\\ \begin{matrix}D = \frac{{C}_{FBL} - R}{{C}_{FBL}} . & ( 11 )\end{matrix}$$

The results are shown in FIG. 23.

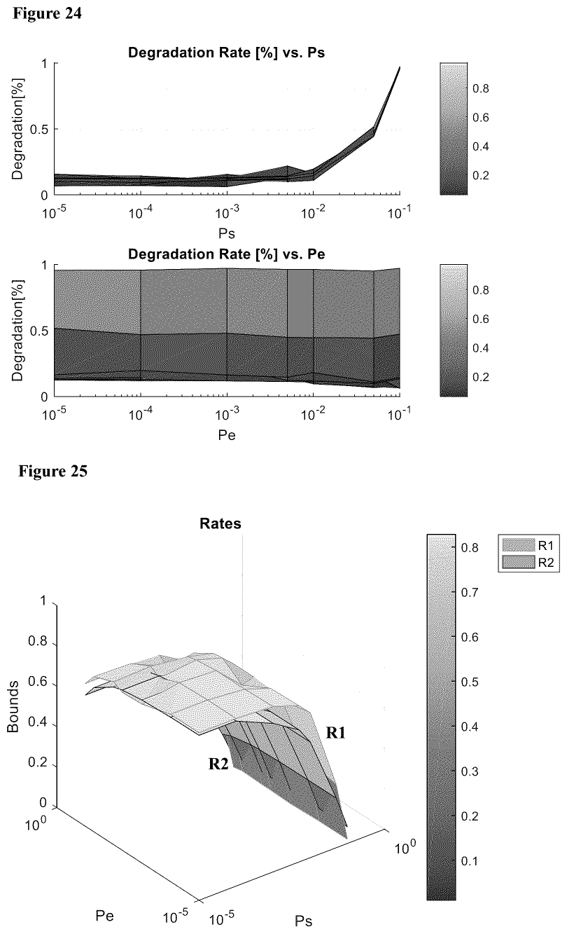

As can be seen in FIG. 24, the encoder-decoder schema is close to the achievable lower bound C_{FBL} with a gap of less than 10%, except for the case of very high substitution rates, namely Pₛ≥0.05. Such high substitution rates are not relevant to our DNA storage problem.

Next, the performance of the coding scheme of the invention was compared with a coding scheme suggested in Shomorony and Heckel (and, for example, used in “Robust chemical preservation of digital information on DNA in silica with error-correcting codes”, Grass et al., Angew Chem Int Ed Engl. 2015 Feb. 16; 54(8):2552-5, herein incorporated by reference in its entirety). Again, the modulating part is not simulated, and the simulation comparison goal is comparing the error correction codes.

In the scheme suggested in Shomorony and Heckel, the entire information is first protected using an outer Reed-Solomon code. Then, the outer encoded information is partitioned into message blocks, and each message block is attached with an identifier. Finally, each message block is protected independently with an inner Reed-Solomon code. Both schemes have a similar first coding step (RS code protecting the whole information chunk) but a different second step (identifiers coding).

The following simulation was performed. The fixed parameters are M=180, L=144, and the varying parameters are Pₑ, Pₛ. The coding rates are R₁, R₂ (the former stands for the coding scheme of the invention, the latter to the one in Shomorony and Heckel), which were optimized to the highest possible rates resulting in no decoding errors (FIG. 25). The ratio

$$\\ \frac{{R}_{1}}{{R}_{2}}$$

is given in Table 3.

TABLE 3
Encoder-Decoder code rate: the ratio between the coding rate of the coding scheme of
the invention and the coding rate of the proposed scheme in Shomorony and Heckel.

| Pe/Ps | 1.00E−05 | 0.0001 | 0.001 | 0.005 | 0.01 | 0.05 | 0.1 |
|---|---|---|---|---|---|---|---|
| 1.00E−05 | 1.014756 | 1.014756 | 1.112934 | 1.183818 | 1.255474 | 1.275 | 0.245455 |
| 0.0001 | 1.017644 | 1.017644 | 1.108159 | 1.177809 | 1.204878 | 1.406072 | 0.24 |
| 0.001 | 0.975086 | 1.019518 | 1.117347 | 1.069921 | 1.260049 | 1.37931 | 0.165517 |
| 0.005 | 0.98609 | 0.98609 | 1.122807 | 1.201754 | 1.283208 | 1.473684 | 0.210526 |
| 0.01 | 1.033254 | 1.04038 | 1.124752 | 1.211581 | 1.229032 | 1.473267 | 0.214286 |
| 0.05 | 1.076023 | 1.068226 | 1.052708 | 1.218522 | 1.337047 | 1.483731 | 0.282353 |
| 0.1 | 1.088571 | 1.088571 | 1.209524 | 1.21125 | 1.31449 | 1.432857 | 0.167143 |

We can see that the scheme of the invention is superior to the schema in Shomorony and Heckel, except for the case of very high substitution rates, namely Pₛ≥0.1 (which are less interesting in DNA storage).

Next, the theoretical computation of the probability of a decoding failure event is considered in both coding schemes. For simplicity, one can assume iid random codewords and a simplified model of iid substitutions errors only (Pₑ=0). A constant bit error probability is denoted by p, resulting in a constant symbol error probability of q=1−(1−p)^{m}, where m is the relevant Reed-Solomon symbol size.

Starting with the first code (of the invention), failure will occur when the decoder is fed with more erroneous symbols than it can fix. Thus,

$$\\ {P}_{failure}^{{}_{} ( 1 )} = Pr ( {N}_{errors} \geq \frac{{n}_{1} - {k}_{1}}{2} + 1 ) = 1 - Pr ( {N}_{errors} \leq \frac{{n}_{1} - {k}_{1}}{2} ) = 1 - {\sum }_{i = 0}^{\frac{{n}_{1} - {k}_{{1}_{}}}{2}} ( \begin{matrix}{n}_{1} \\ i\end{matrix} ) {{q}_{1}^{{}_{} i} ( 1 - {q}_{1} )}^{{n}_{1} - i}$$

Where all parameters refer to the Reed-Solomon code of the first schema, namely m₁ is the symbol size, k₁ is the information size in symbols, and n₁ is the codeword length .q₁=1−(1−p)^{m}^{₁} is the symbol error probability.

As for the second code, which consists of an outer and inner code, the event of decoding failure will occur when the outer code is fed with more erroneous symbols than it can fix. An inner code block decoding failure causes an incorrect outer code symbol. The probability of an inner code failure event is

$$\\ {P}_{failure}^{{}_{} ( 2 , inner )} = Pr ( {N}_{errors} \geq \frac{{N}_{2} - {K}_{2}}{2} + 1 ) = 1 - Pr ( {N}_{errors} \leq \frac{{N}_{2} - {K}_{2}}{2} ) = 1 - {\sum }_{i = 0}^{\frac{{N}_{2} - {K}_{{2}_{}}}{2}} ( \begin{matrix}{N}_{2} \\ i\end{matrix} ) {{Q}_{2}^{{}_{} i} ( 1 - {Q}_{2} )}^{{N}_{2} - i} {=}^{△} P$$

where all parameters refer to the inner Reed-Solomon code of the second schema, namely m₂ is the symbol size, K₂ is the information size in symbols, and N₂ is the codeword length .Q₂=1−(1−p)^{m}^{₂} is the symbol error probability.

Inner code decoding failure is causing an error of

$$\\ \frac{L}{{m}_{1}}$$

outer code symbols (each inner codeword, protecting a single message block, consists of

$$\\ \frac{L}{{m}_{1}}$$

outer code symbols). Thus, the event of an outer code decoding failure is

$$\\ {P}_{failure}^{{}_{} ( 2 )} = Pr ( {N}_{errors} \geq \frac{{n}_{2} - {k}_{2}}{2} + 1 ) = 1 - Pr ( {N}_{errors} \leq \frac{{n}_{2} - {k}_{2}}{2} ) = 1 - {\sum }_{i = 0}^{\frac{{n}_{2} - {k}_{{2}_{}}}{2 \frac{{}^{} L}{{m}_{1}}}} ( \begin{matrix}\frac{{n}_{2}}{\frac{L}{{m}_{1}}} \\ i\end{matrix} ) {{P}^{{}_{} i} ( 1 - P )}^{{n}_{2} / ( \frac{L}{{m}_{1}} ) - i} = 1 - {\sum }_{i = 0}^{\frac{( {n}_{2} - {k}_{2} ) {m}_{{1}_{}}}{2 L}} ( \begin{matrix}\frac{{n}_{2} {m}_{1}}{L} \\ i\end{matrix} ) {{P}^{{}_{} i} ( 1 - P )}^{\frac{{n}_{2} {m}_{{1}_{}}}{L} - i}$$

where all parameters refer to the outer Reed-Solomon code of the second schema, and P=P_{failure}^{(2,inner)} is given above. One can assume that the symbol size is the same as the first schema, m₁.

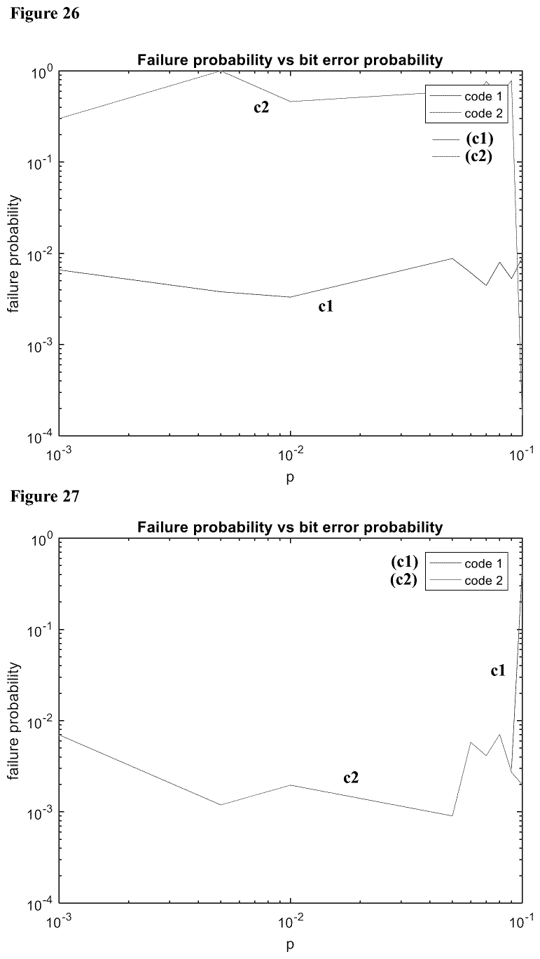

Now one can numerically compare P_{failure}⁽¹⁾, P_{failure}⁽²⁾ for the relevant parameters achieving the same coding rates in both schemas. First, one fixes P_{failure}⁽¹⁾≈5·10⁻³, finding the coding rate achieving this failure probability, fixes the same rate for the second schema, and calculates P_{failure}⁽²⁾ (FIG. 26). One sees that the first schema has a much better performance compared to the second, except for the case of bit error rate greater than 0.09.

Second, one can fix P_{failure}⁽²⁾≈5·10⁻³, find the coding rate achieving this failure probability, fix the same rate for the first schema, and calculate P_{failure}⁽¹⁾ (FIG. 27). Again, one sees that the first schema has a much better performance compared to the second (all failure probabilities of the first schema are numerically zero except the two last simulated bit error probabilities), again except in the case of bit error rate greater than 0.09.

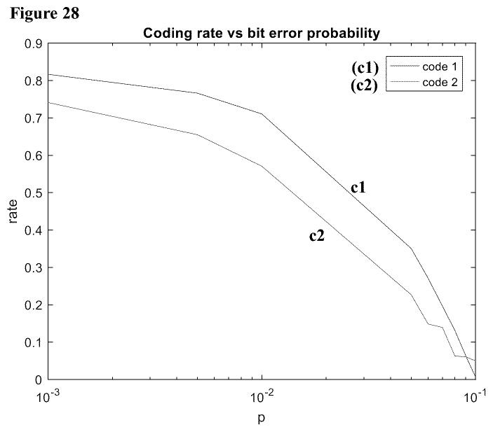

Last, one can fix P_{failure}⁽¹⁾=P_{failure}⁽²⁾≈5·10⁻³, and find the rates achieving this failure probability for each schema (FIG. 28). Again, one sees that the first schema can achieve higher coding rate for all bit error rates, except of the case of bit error rate greater than 0.09.

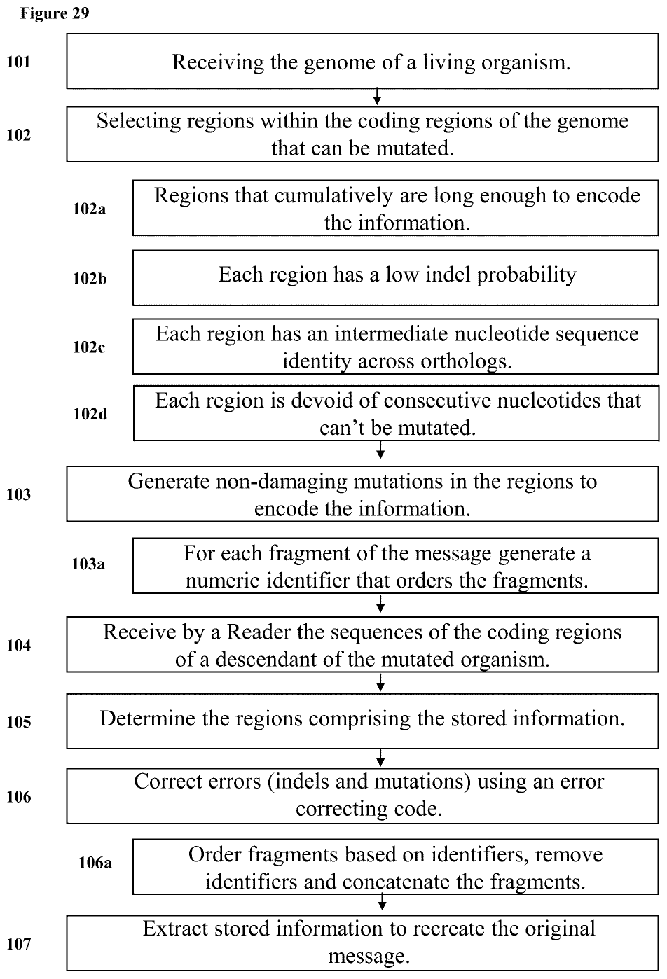

The overall method of encoding information in a living organism is summarized in FIG. 29. At step 101, the genome of an organism into which the information is going to be stored is received. Next, at step 102, regions within the coding regions of the genome are selected which meet the desired criteria. These criteria include: the regions being cumulatively of a sufficient length to encode the full message with identifiers that give the order to the various fragments of the information (102a); a low indel probability (102b); and an intermediate nucleotide sequence identity (102c). Optionally, the regions are selected such that they are devoid of two consecutive nucleotides which cannot be mutated without substantially diminishing the health of the organism (102d). Mutations are then generated in the regions selected in step 102 (step 103). The mutations are convertible to the information. That is the mutations encode the information. As used herein, the terms “convertible to” and “encoding” are used synonymously and interchangeably. Generally, bits of data are converted into nucleotides. The mutations generated are not substantially detrimental to the health of the organism. Detrimental mutations would lead to the death of the organism overtime and thereby erasure of part or all of the information. Identifiers can be optionally included which identify the order of the fragments that produce the whole information (step 103a).

FIG. 29 also includes optional steps related to decoding the information at a later time point. The decoder does not necessarily have the information of the location of the fragments but does have the coding matrix used for encoding the information in the first place (the mutations made). At step 104, the Reader receives the sequences of the coding regions of the muted organism's descendants. This can be done by sequencing the genome of the organism via methods such as deep sequencing, whole exosome sequencing, next generation sequencing and the like. The fragments are identified (105) At step 106, the Reader corrects any error introduced into the fragments, including indels and point mutations. Optionally, the fragments are put in order based on the identifier sequence if present. The identifiers are removed, and the fragments are concatenated (step 106a). Finally, at step 107 the information is extracted from the genomic fragment.

### Example 14: Numerical Simulation

In order to evaluate the results, a numerical simulation was performed. First, a set of DNA sites optimal for storage needs to be chosen. For that, unsuitable genes were filtered out. Following definitions provided hereinabove, the criteria for a suitable gene (or, more precisely, a site) for DNA storage are as follows.

First, a long sequence of codons with a low and correlative indel probability is found. Mathematically, one is looking for a site S such that (27) gives a value smaller than some threshold and when |S| is big enough. The reason for that is to minimize the chance of microorganism erasure or an indel in some part of the site of interest. A representative example is the site in the ALA1 gene defined in Example 11, following Error! Reference source not found. A-B, in which all codons are associated with indel probability lower than a threshold. Also, the site length is long enough (about 300 codons).

Next, a mid-range average identity probability is selected. Mathematically, one is looking for a site S such that (26) gives a value smaller than some high threshold and bigger than some low threshold. The reason for that is the following. On the one hand, we would prefer a high identity probability in order to avoid mutations. On the other hand, we would prefer a low identity so that changes made by our storage would not be too harmful to the creature. Again, Error! Reference source not found. 15A gives a good idea for a site of interest, with a mid-range identity probability of about 0.7.

The last criterion at this point is that the size of the average subset of allowed codons changes is large enough. Mathematically, it is required that (29) gives a value higher than some threshold when the dictionary D is chosen according to (28). This way, a change of codon into another codon is one that appeared in enough orthologs, which is an indicator that evolution prefers such a change. FIG. 16 is an example of the size of the subset along the site of interest. For a threshold of 0.05, meaning allowing a change into a codon that appeared in at least 5% of aligned orthologs, one sees that the average subset size is about 4.5, which we found to be a relatively large subset size, allowing coding using the Linear Random Subsets code construction designed hereinabove.

The site filtering constraints are now summarized. A site S with at least |S|=200 codons, with indel probability (27) not exceeding 0.05, average identity probability (26) in the range 0.55-0.75, and average subset size (29) (where the dictionary is chosen according to (28)) of at least four codons. Also, site sequences with more than two consecutive DNA locations in which no changes at all are possible ((28) results in a value smaller than 1) are not allowed in order to avoid situations of no possible encoding using the Linear Random Subsets method. Imposing these constraints over 3960 different S. cerevisiae genes with full alignment data, came up with 40 possible sites in 38 different genes.

Next, it was decided to store over M=64 synthesized sites. With a maximal site length of |S|=200 codons, the message block size was fixed in bits (number of bits stored in each synthesized site), a quantity defined by L. The relation between the site length |S|, the message block length L, and the Linear Random Subsets Code (LRSC) inner matrix H_{q}_{_{LRSC}}_{×n}_{_{LRSC}} is

$$\\ \vert S \vert = \frac{L}{2} \frac{{n}_{LRSC}}{{q}_{LRSC}} ,$$

as the whole message block is to be encoded over the whole site using LRSC, operating over the nucleotide Galois Field GF(4), modulating each q_{LRSC} nucleotide from the message block (out of all L/2) into n_{LRSC} nucleotides. It was decided to work with n_{LRSC}=15, q_{LRSC}=2, as a larger n_{LRSC} ended up too complexity demanding for this simulation, and q_{LRSC} was the maximal integer allowing coding without failures among all tested sites. Fixing L=144 bits came up with |S|=540 nucleotides or 180 codons.

The LRSC inner matrix used in the simulation is

$$H = [ \begin{matrix}AAAACCCCGGGGTTT \\ ACGTACGTACGTACG\end{matrix} ] . \qquad ( SEQ ID NO : 6 - 7 )$$

At first glance, it seems like a non-ideal matrix for encoding using the LRSC, as the first column is the zero column, which actually prevents the first nucleotide in each subsite from participating in the linear equations (recall from above that a subsite is defined as a sequence of n_{LRSC} nucleotides which participate in the same algebraic equation for inner encoding). But, it was found empirically that such a choice is actually advantageous, as sometimes a codon appears with a very small subset size allowed for changes (28), in which case it should be kept out of the algebraic equation.

Each identifier was chosen to be k_{id}=log M=6 bits long, with a coding rate of r_{id}=0.5, such that the encoded identifier length is n_{id}=12 bits. The Reed-Solomon symbol length is chosen as the minimal possible, m_{RS,id}=3 bits. The reason for that code rate is to allow maximal protection to the identifiers, as a decoding error of it will be a loss of the entire message block.

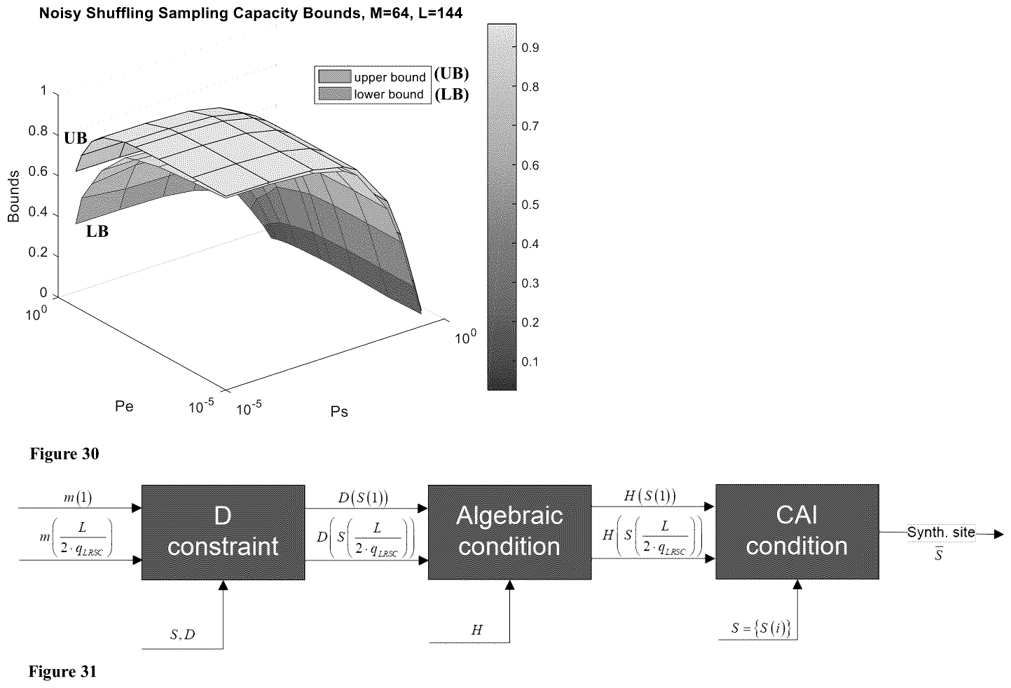

The code-word length of the information Reed-Solomon code is, therefore, n=M(L−n_{id})=8448 bits, as it covers all the message blocks together, without the identifiers. For such code-word length, the symbol length we chose was m_{RS}=12 bits, and a shortened Reed-Solomon is used. It should be noted that the symbol length could be shorter (10 bits), which is better in terms of code rate performance, but m_{RS}=12 was chosen as it enables more flexibility for bigger code-word length (up to (2^{m}^{_{RS}}−1)m_{RS}=49140 bits). The number of information bits is k=r·n, where r is the code rate of the encoder-decoder part of the coding scheme. In order to determine r, one first calculates the bounds in (9) for M=64, L=144, ε=10⁻⁶. The results are given in FIG. 30.

Note that, in comparison to f, the gap between the bounds is higher, a consequence of the lower value of M here.

Now, for given error probabilities Pₑ, Pₛ, one can adjust r in a way that will end up without any simulated decoding failures, with a good starting point of r which equals the lower bound value. The actual metric of such an experiment is the amount of erroneous Reed-Solomon symbols at the decoder, compared to the decoding capability of the code, which equals d=n_{RS}−k_{RS}, where n_{RS}, k_{RS} are the code-word length and the message length of the code, measured in symbols.

At this point, the error probabilities are fixed to Pₛ=0.005, Pₑ=0.1. These are relatively high error rates for a real biological environment. It was chosen to focus on such high error rates for both maximal data protection and as a preparation for an in vivo experiment, which includes a mutant strain simulating a high mutation rate.

After fixing Pₛ=0.005, Pₑ=0.1, the experimental test was evaluated to find the highest value of r without any decoding failures. The value found was r=0.655·R*_{FBL}(M=64, L=144, ε=10⁻⁶), where R^{*}_{FBL} is the lower bound in (9). Such a value seems like a big gap from the achievability bound, but the following reason reveals why. Note that the Random Subset Channel plays a role here, as an outer error correcting code was not designed for the Linear Random Subset code, as the construction demands. Actually, the Reed Solomon code protecting the information from message block erasures and substitutions, is also protecting it from cases in which the Linear Random Subsets encoder fails to decode. As mentioned, the actual Linear Random Subsets code parameters were fixed to a code rate of

$$\\ \frac{{q}_{LRSC}}{{n}_{LRSC}} \cdot 2 \frac{bits}{nucleotides} \cdot 3 \frac{nucleotides}{codon} = 0.8 \frac{bits}{codon} ,$$

which is appropriate for an average subset size of about 4 legal codon changes, as suggested by FIG. 14.

Lastly, the allowed codon changes dictionary D used for the Linear Random Subsets code was employed. Three different dictionaries were designed, each for different reason.

The first dictionary is the most conservative one, allowing only synonymous changes of codons. Also, a codon change is allowed only if the codon has appeared in at least 5% among all aligned orthologs in the same location, meaning it is favorable by evolution in some sense. The second dictionary is the most liberal one, allowing both synonymous and non-synonymous changes of codons. For synonymous changes, the minimal appearances threshold was set to 5% as before, and for non-synonymous changes, the threshold was set to 25%. The reason for that is to allow such a change, which changes the protein ingredient of the microorganism, only if evolution favorites such a change in many cases. The third dictionary, which is a middle ground, is designed exactly as the second dictionary, just with a higher threshold of synonymous changes equals 0.08. The reason for that is to balance the allowed changes to the point of highly favorable changes of evolution but still allow both synonymous and non-synonymous changes of codons. All simulations were performed.

The abstract of an article summarizing this work was taken as the information message, it was transformed into bits using ASCII encoding (with 1 byte per character), and it was truncated to the message length in bits, which in this case is k=r·n=0.65·R^{*}_{FBL}(M=64, L=144, ε=10⁻⁶)·n=0.655·0.6245·8448=3444. Then, the encoder took the information bits, protected it with a shortened Reed-Solomon code (with message length of k=3444 bits, code-word length of n=8448 and symbol size m=12 bits), split the code-word into M=64 chunks of L−n_{id}=n/M=132 bits each, and attached each chunk with an encoded identifier of length n_{id}=12 bits (which is itself a Reed-Solomon code-word, generated by a shortened Reed-Solomon encoder with message length of k_{id}=log M=6 bits and symbol size of m_{RS}=3 bits), to generate all M=64 message blocks of length L=144 bits each. The modulator operated overall M=64 message blocks and generated synthesized sites of length

$$\\ \vert S \vert = \frac{L}{2} \frac{{n}_{LRSC}}{{q}_{LRSC}} = 540$$

nucleotides (or 180 codons) each, one site per message block. Each modulation operated according to the Linear Random Subsets Encoder, with LRSC inner matrix

$$H = [ \begin{matrix}AAAACCCCGGGGTTT \\ ACGTACGTACGTACG\end{matrix} ] , \qquad ( SEQ ID NO : 6 - 7 )$$

sub-sites size n_{LRSC}=15 nucleotides, each sub-site modulating q_{LRSC}=2 information nucleotides (or 4 bits). Also, the legal codon changes dictionary used is one of the three dictionaries defined above. The actual Linear Random Subsets encoder was implemented according to FIG. 31, minimizing the Codon Adaptation Index.

Next, the Living Organism Channel was simulated. The subset constraints of the Random Subsets Channel are always obeyed, so the actual channel simulated is the Noisy Shuffling Sampling Channel. First, each synthesized site is erased with a probability of Pₑ=0.1, in an iid manner. Then, all sites are permuted randomly. Finally, each nucleotide suffers from substitution with a probability of P_{S}=0.005. The demodulator accepts N≤M sites, performing Linear Random Subsets decoding with the LRSC inner matrix H, thus transforming each read site into a sequence of L/2 nucleotides, and finally transforms them into L bits. The decoder is fed with N message blocks, and its first mission is to sort them back in their original order. For that purpose, it decodes the identifiers in a Maximum Likelihood fashion, as explained hereinabove, exploiting the information that it expects to get all integers in the range {0, . . . , M−1} and thus map each identifier to the closet integer in {0, . . . , M−1} in the sense of hamming distance. After the messages are sorted, the identifiers are disregarded, zero bits are inserted instead of erased message blocks, and a Reed-Solomon decoder is evaluated over all the corrupted data. At this point, the stored data was checked to see if it is reconstructed successfully.

The following tables summarize the results for all three dictionaries and 40 possible coding sites that passed the initial filtering described hereinabove. All sites in each gene are 540 nucleotides (180 AA) long, starting at the nucleotide given in the second column. Code rate is the actual number of information bits stored over one codon, while the achievable rate is the product of the lower bound of the Random Subsets channel capacity and the lower bound of the Noisy Shuffling Sampling channel capacity. The number of Reed-Solomon symbol errors indicating how much corrupted Reed-Solomon symbols were corrupted at the receiver, and the error correction capability of the code is given too. The average modulator failure rate meaning the fractional number of cases in which no encoding was possible for some sub-site using the Linear Random Subsets method. The CAI (codon adaptation index) is given before and after the writing process, and also the average number of cases in which an amino acid was changed (non-synonymous change).

TABLE 4
Simulation results, first dictionary (allowing only synonymous changes
with at least 5% appearance among all aligned orthologs).

| | first | | | number | RS | average | | | average |
|---|---|---|---|---|---|---|---|---|---|
| | nt in | code | achievable | of RS | correction | modulator | CAI | CAI | number of |
| gene name | site | rate | rate | errors | ability | failure rate | original | new | AA changed |
| CDC48 | 583 | 0.29896 | 0.330962 | 359 | 417 | 0.012153 | 0.85207 | 0.69716 | 0 |
| ACS2 | 106 | 0 | 0.330962 | 1067 | 417 | 0.121528 | 0.82959 | 0.73265 | 0 |
| LEU4 | 238 | 0 | 0.330962 | 480 | 417 | 0.05816 | 0.79706 | 0.70134 | 0 |
| LEU9 | 235 | 0 | 0.330962 | 516 | 417 | 0.064236 | 0.79633 | 0.70163 | 0 |
| PFK1 | 2011 | 0 | 0.330962 | 704 | 417 | 0.082465 | 0.80976 | 0.68868 | 0 |
| SSE1 | 586 | 0 | 0.330962 | 471 | 417 | 0.055556 | 0.83573 | 0.73854 | 0 |
| RPT3 | 223 | 0.29896 | 0.537033 | 382 | 417 | 0 | 0.7805 | 0.66025 | 0 |
| RPT2 | 307 | 0.29896 | 0.330962 | 317 | 417 | 0 | 0.77179 | 0.6554 | 0 |
| ALA1 | 1561 | 0.29896 | 0.330962 | 311 | 417 | 0.012587 | 0.84025 | 0.7239 | 0 |
| WRS1 | 112 | 0.29896 | 0.330962 | 401 | 417 | 0.013889 | 0.77474 | 0.68148 | 0 |
| HCA4 | 115 | 0 | 0.537033 | 436 | 417 | 0.013889 | 0.76225 | 0.64034 | 0 |
| HCA4 | 775 | 0 | 0.330962 | 534 | 417 | 0.025608 | 0.80491 | 0.68137 | 0 |
| RPG1 | 1648 | 0 | 0 | 1282 | 417 | 0.527344 | 0.78251 | 0.72914 | 0 |
| PIL1 | 151 | 0 | 0.330962 | 566 | 417 | 0.057726 | 0.79093 | 0.70405 | 0 |
| LSP1 | 151 | 0 | 0.330962 | 496 | 417 | 0.044271 | 0.76853 | 0.67753 | 0 |
| EFT2 | 1123 | 0 | 0.330962 | 543 | 417 | 0.074653 | 0.83827 | 0.70761 | 0 |
| EFT1 | 1123 | 0 | 0.330962 | 527 | 417 | 0.074653 | 0.83827 | 0.70761 | 0 |
| SUL2 | 1144 | 0 | 0.330962 | 718 | 417 | 0.126736 | 0.78 | 0.68491 | 0 |
| SUL1 | 1096 | 0 | 0.330962 | 683 | 417 | 0.09809 | 0.74766 | 0.64621 | 0 |
| RPD3 | 304 | 0 | 0.330962 | 442 | 417 | 0.02691 | 0.72443 | 0.62738 | 0 |
| SSZ1 | 751 | 0 | 0.330962 | 735 | 417 | 0.128472 | 0.82813 | 0.72984 | 0 |
| NVJ2 | 847 | 0 | 0.330962 | 945 | 417 | 0.183594 | 0.78439 | 0.68634 | 0 |
| SSE2 | 586 | 0 | 0.330962 | 626 | 417 | 0.082899 | 0.76463 | 0.6741 | 0 |
| PUF4 | 1636 | 0 | 0 | 1309 | 417 | 0.878906 | 0.76729 | 0.71033 | 0 |
| DPH5 | 121 | 0.29896 | 0.330962 | 408 | 417 | 0.008247 | 0.78969 | 0.67265 | 0 |
| NDE2 | 289 | 0 | 0.330962 | 718 | 417 | 0.137587 | 0.70239 | 0.61386 | 0 |
| MGM1 | 607 | 0 | 0 | 1309 | 417 | 0.861111 | 0.75877 | 0.6729 | 0 |
| SSQ1 | 868 | 0 | 0.330962 | 932 | 417 | 0.056858 | 0.77885 | 0.67194 | 0 |
| TOR1 | 1693 | 0 | 0.330962 | 1071 | 417 | 0.13151 | 0.7412 | 0.64091 | 0 |
| TOR2 | 1720 | 0 | 0.330962 | 1286 | 417 | 0.157118 | 0.76207 | 0.67047 | 0 |
| GCN1 | 4012 | 0 | 0.330962 | 681 | 417 | 0.073351 | 0.79799 | 0.68779 | 0 |
| GCN1 | 4741 | 0 | 0.330962 | 630 | 417 | 0.115451 | 0.77445 | 0.6757 | 0 |
| SBE22 | 1222 | 0 | 0.330962 | 526 | 417 | 0.068142 | 0.78162 | 0.68113 | 0 |
| RRP3 | 766 | 0.29896 | 0.330962 | 374 | 417 | 0.021267 | 0.76556 | 0.65089 | 0 |
| MLH1 | 19 | 0 | 0.330962 | 552 | 417 | 0.056858 | 0.73254 | 0.62657 | 0 |
| RIX7 | 1486 | 0 | 0.330962 | 425 | 417 | 0.008681 | 0.78315 | 0.64808 | 0 |
| VPS17 | 679 | 0 | 0.330962 | 1170 | 417 | 0.296875 | 0.7414 | 0.64533 | 0 |
| SMC3 | 295 | 0 | 0.330962 | 888 | 417 | 0.230035 | 0.76515 | 0.6596 | 0 |
| DNF3 | 3493 | 0 | 0.330962 | 580 | 417 | 0.074653 | 0.74339 | 0.63842 | 0 |
| YCF1 | 1924 | 0 | 0.330962 | 463 | 417 | 0.039063 | 0.79825 | 0.68718 | 0 |

TABLE 5
simulation results, second dictionary (allowing synonymous changes with at least 5% appearance
and non-synonymous changes with at least 25% appearance among all aligned orthologs).

| | first | | | number | RS | average | | | average |
|---|---|---|---|---|---|---|---|---|---|
| | nt in | code | achievable | of RS | correction | modulator | CAI | CAI | number of |
| gene name | site | rate | rate | errors | ability | failure rate | original | new | AA changed |
| CDC48 | 583 | 0.29896 | 0.330962 | 329 | 417 | 0.012153 | 0.85207 | 0.69935 | 1.0469 |
| ACS2 | 106 | 0 | 0.330962 | 728 | 417 | 0.060764 | 0.82959 | 0.72554 | 5.6875 |
| LEU4 | 238 | 0.29896 | 0.330962 | 378 | 417 | 0.015191 | 0.79706 | 0.70234 | 6.2188 |
| LEU9 | 235 | 0.29896 | 0.330962 | 392 | 417 | 0.032118 | 0.79633 | 0.70744 | 5.1875 |
| PFK1 | 2011 | 0 | 0.330962 | 452 | 417 | 0.019965 | 0.80976 | 0.69427 | 7.3125 |
| SSE1 | 586 | 0.29896 | 0.330962 | 381 | 417 | 0 | 0.83573 | 0.73885 | 9.6719 |
| RPT3 | 223 | 0.29896 | 0.537033 | 350 | 417 | 0 | 0.7805 | 0.66795 | 4.9688 |
| RPT2 | 307 | 0.29896 | 0.537033 | 310 | 417 | 0 | 0.77179 | 0.66228 | 5.5156 |
| ALA1 | 1561 | 0.29896 | 0.330962 | 354 | 417 | 0 | 0.84025 | 0.73393 | 6.7813 |
| WRS1 | 112 | 0.29896 | 0.330962 | 349 | 417 | 0 | 0.77474 | 0.68726 | 4.875 |
| HCA4 | 115 | 0.29896 | 0.537033 | 416 | 417 | 0.013889 | 0.76225 | 0.64227 | 6.0313 |
| HCA4 | 775 | 0.29896 | 0.330962 | 336 | 417 | 0 | 0.80491 | 0.6867 | 7.4531 |
| RPG1 | 1648 | 0 | 0.330962 | 774 | 417 | 0.140191 | 0.78251 | 0.69254 | 26 |
| PIL1 | 151 | 0.29896 | 0.330962 | 353 | 417 | 0.008247 | 0.79093 | 0.70377 | 5.8281 |
| LSP1 | 151 | 0.29896 | 0.537033 | 328 | 417 | 0 | 0.76853 | 0.68296 | 5.9063 |
| EFT2 | 1123 | 0 | 0.330962 | 550 | 417 | 0.06467 | 0.83827 | 0.71671 | 3.9688 |
| EFT1 | 1123 | 0 | 0.330962 | 560 | 417 | 0.06467 | 0.83827 | 0.71671 | 3.9688 |
| SUL2 | 1144 | 0 | 0.330962 | 467 | 417 | 0.033854 | 0.78 | 0.68719 | 9.9688 |
| SUL1 | 1096 | 0 | 0.537033 | 451 | 417 | 0.048177 | 0.74766 | 0.64673 | 7.2813 |
| RPD3 | 304 | 0.29896 | 0.537033 | 315 | 417 | 0.013021 | 0.72443 | 0.6262 | 2.2031 |
| SSZ1 | 751 | 0 | 0.330962 | 476 | 417 | 0.032118 | 0.82813 | 0.72173 | 11.391 |
| NVJ2 | 847 | 0 | 0.330962 | 581 | 417 | 0.031684 | 0.78439 | 0.69304 | 12.844 |
| SSE2 | 586 | 0 | 0.330962 | 478 | 417 | 0.012587 | 0.76463 | 0.67308 | 10.234 |
| PUF4 | 1636 | 0 | 0.330962 | 995 | 417 | 0.130208 | 0.76729 | 0.72453 | 51.609 |
| DPH5 | 121 | 0.29896 | 0.537033 | 315 | 417 | 0 | 0.78969 | 0.67723 | 4.9375 |
| NDE2 | 289 | 0 | 0.330962 | 622 | 417 | 0.085069 | 0.70239 | 0.6194 | 9.3438 |
| MGM1 | 607 | 0 | 0.330962 | 1044 | 417 | 0.167969 | 0.75877 | 0.67362 | 48.234 |
| SSQ1 | 868 | 0 | 0.330962 | 640 | 417 | 0.042969 | 0.77885 | 0.67956 | 5.4531 |
| TOR1 | 1693 | 0.29896 | 0.537033 | 385 | 417 | 0.030382 | 0.7412 | 0.6466 | 14.516 |
| TOR2 | 1720 | 0 | 0.537033 | 577 | 417 | 0.039497 | 0.76207 | 0.67205 | 12.922 |
| GCN1 | 4012 | 0 | 0.537033 | 567 | 417 | 0.027778 | 0.79799 | 0.69637 | 10 |
| GCN1 | 4741 | 0.29896 | 0.537033 | 361 | 417 | 0.007813 | 0.77445 | 0.67921 | 11.188 |
| SBE22 | 1222 | 0.29896 | 0.330962 | 379 | 417 | 0.019097 | 0.78162 | 0.68873 | 7.7344 |
| RRP3 | 766 | 0.29896 | 0.537033 | 382 | 417 | 0.013021 | 0.76556 | 0.64998 | 6.2813 |
| MLH1 | 19 | 0 | 0.330962 | 431 | 417 | 0.022569 | 0.73254 | 0.62581 | 7.1406 |
| RIX7 | 1486 | 0.29896 | 0.537033 | 358 | 417 | 0.001736 | 0.78315 | 0.65749 | 6.0781 |
| VPS17 | 679 | 0 | 0.330962 | 765 | 417 | 0.154948 | 0.7414 | 0.655 | 14.141 |
| SMC3 | 295 | 0 | 0.330962 | 657 | 417 | 0.086806 | 0.76515 | 0.65407 | 16.547 |
| DNF3 | 3493 | 0 | 0.330962 | 508 | 417 | 0.053385 | 0.74339 | 0.6361 | 4.9375 |
| YCF1 | 1924 | 0.29896 | 0.537033 | 328 | 417 | 0.014323 | 0.79825 | 0.69211 | 4.375 |

TABLE 6
simulation results, third dictionary (allowing synonymous changes with at least 8% appearance
and non-synonymous changes with at least 25% appearance among all aligned Orthologs).

| | first | | | number | RS | average | | | average |
|---|---|---|---|---|---|---|---|---|---|
| | nt in | code | achievable | of RS | correction | modulator | CAI | CAI | number of |
| gene name | site | rate | rate | errors | ability | failure rate | original | new | AA changed |
| CDC48 | 583 | 0.29896 | 0.330962 | 376 | 417 | 0.02691 | 0.85207 | 0.70136 | 1.40625 |
| LEU4 | 238 | 0 | 0.330962 | 639 | 417 | 0.033854 | 0.79706 | 0.70646 | 6.8125 |
| LEU9 | 235 | 0 | 0.330962 | 431 | 417 | 0.036892 | 0.79633 | 0.70282 | 6.48438 |
| SSE1 | 586 | 0 | 0.330962 | 485 | 417 | 0.052083 | 0.83573 | 0.73301 | 11.4063 |
| RPT3 | 223 | 0.29896 | 0.537033 | 405 | 417 | 0 | 0.7805 | 0.66684 | 6.21875 |
| RPT2 | 307 | 0.29896 | 0.330962 | 354 | 417 | 0 | 0.77179 | 0.6644 | 7.0625 |
| ALA1 | 1561 | 0.29896 | 0.330962 | 410 | 417 | 0.013889 | 0.84025 | 0.7324 | 9.09375 |
| WRS1 | 112 | 0 | 0.330962 | 435 | 417 | 0.035156 | 0.77474 | 0.68484 | 6.20313 |
| HCA4 | 115 | 0 | 0.330962 | 631 | 417 | 0.024306 | 0.76225 | 0.6269 | 8.39063 |
| HCA4 | 775 | 0.29896 | 0.330962 | 389 | 417 | 0.027778 | 0.80491 | 0.67484 | 11.0625 |
| PIL1 | 151 | 0 | 0.330962 | 429 | 417 | 0.042969 | 0.79093 | 0.70106 | 7.04688 |
| LSP1 | 151 | 0 | 0.330962 | 427 | 417 | 0.03559 | 0.76853 | 0.678 | 5.96875 |
| RPD3 | 304 | 0 | 0.330962 | 505 | 417 | 0.02474 | 0.72443 | 0.61912 | 2.78125 |
| DPH5 | 121 | 0 | 0.330962 | 545 | 417 | 0.03342 | 0.78969 | 0.68708 | 7.07813 |
| TOR1 | 1693 | 0 | 0.330962 | 677 | 417 | 0.092014 | 0.7412 | 0.64048 | 14.8594 |
| GCN1 | 4741 | 0.29896 | 0.330962 | 403 | 417 | 0.007813 | 0.77445 | 0.67131 | 14.3281 |
| SBE22 | 1222 | 0 | 0.330962 | 572 | 417 | 0.059896 | 0.78162 | 0.68499 | 9.9375 |
| RRP3 | 766 | 0 | 0.330962 | 424 | 417 | 0.025608 | 0.76556 | 0.64072 | 8.32813 |
| RIX7 | 1486 | 0 | 0.330962 | 685 | 417 | 0.052083 | 0.78315 | 0.6587 | 8.64063 |
| YCF1 | 1924 | 0 | 0.330962 | 514 | 417 | 0.057726 | 0.79825 | 0.70056 | 6.28125 |

The Table 6 includes only the sites with non-zero code rates in Table 5.

The achievable rate in all tables above refers to the term R^{*}_{FBL} (M=64, L=144, ε=10⁻⁶)·R_{CRSC}(K), which is the product of the achievable Finite Block-Length coding rate of the Noisy Shuffling Sampling channel, and the achievable coding rate of the Completely Random Subsets channel, with appropriate parameters (M=64, L=144, ε=10⁻⁶ are fixed, and K is calculated according to the actual dictionary estimated for each site, with observed values of K=3,4 in all cases).

As one can see, the sites in genes CDC48, RPT3,RPT2 and ALA′ can be used with all dictionary options, all with code rate very close to the achievable rate, except for RPT3 in which further parameters optimization (such as the higher value of n_{LRSC}) can be performed.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of storing information in a living organism, the method comprising:
a. receiving a genomic sequence of said living organism;
b. selecting a fragment of a coding region within said received genomic sequence in which to store said information, wherein said selecting comprises:
i. estimating indel probability across coding regions of said genomic sequence and selecting fragments of said coding regions with a probability below a predetermined threshold for all nucleotides within said fragment;
ii. receiving coding regions of orthologs of said organism and selecting fragments of said coding regions of said living organism with an intermediate nucleotide sequence identity probability across said orthologs; and
iii. selecting a fragment of a coding region that was selected in both (i) and (ii); and
c. inducing genetic mutations in said selected fragment of a coding region in a cell of said living organism such that said genetic mutations are convertible to said information, wherein said mutations are not substantially detrimental to the health of said living organism;
thereby storing information in a living organism.

2. The method of claim 1, wherein said fragment is between 150-350 codons in length.

3. (canceled)

4. The method of claim 1, wherein said living organism is a single celled organism, optionally wherein said living organism is a bacterium

5. (canceled)

6. The method of claim 1, wherein said selected fragment comprises an indel probability variance below a predetermined threshold for all nucleotides within said fragment, optionally wherein said indel probability variance threshold is 0.05.

7. (canceled)

8. The method of claim 1, wherein at least one of:
a. coding regions from at least 100 orthologs are received;
b. an intermediate identity probability is between 0.55 and 0.75.

9. (canceled)

10. The method of claim 1, wherein said not substantially detrimental mutation is a synonymous mutation.

11. The method of claim 1, wherein said not substantially detrimental mutation is a mutation to a codon that appears within at least 5% of orthologs.

12. (canceled)

13. The method of claim 1, wherein said fragment is devoid of at least 2 consecutive nucleotides which cannot be mutated without being substantially detrimental to the health of said living organism.

14. The method of claim 1, wherein said inducing genetic mutations comprises optimizing for said living organism the codon adaptation index (CAI) within said fragment, optionally wherein an optimized CAI is a CAI with the smallest change from the unmutated fragment sequence.

15. (canceled)

16. The method of claim 1, further comprising producing genetic mutations convertible to a numeric identifier which identifies the order in which the information was stored in a plurality of fragments.

17. The method of claim 16, wherein a first or second codon of said fragment is mutated to include information of said numeric identifier. wherein said method comprises storing information in a plurality of different organisms and wherein said numeric identifier identifies the order of the information across said plurality of different organisms or both

18. (canceled)

19. The method of claim 1, wherein said stored information retains at least 90% fidelity after at least 50 generations of said genetically mutated living organism, said information is encoded into said mutations using an error correcting code or both

20. (canceled)

21. The method of claim 19, wherein at least one of:
a. said error correcting code is a Reed-Solomon (RS) code;
b. said information is encoded using a modulation algorithm; and
c. said fragment comprises mutations convertible to a numeric identifier and wherein said mutations convertible to said information and said mutations convertible to said numeric identifier are encoded separately.

22. (canceled)

23. (canceled)

24. The method of claim 1, further comprising reading said stored information, wherein said reading comprises:
d. receiving by a reader, sequences of coding regions of said genetically mutated living organism or a descendant of said genetically mutated living organism;
e. identifying the fragments comprising the stored information;
f. correcting any indels and any point mutations within said fragments that arose since said inducing; and
g. extracting said information from said induced genetic mutations.

25. The method of claim 24, wherein said identifying fragments comprises comparison to the native genome of said living organism or said fragment locations are known and said identifying comprises locating said fragments within said received sequences.

26. (canceled)

27. The method of claim 24, further comprising between step (e) and (f) a step comprising reading a numeric identifier in a plurality of fragments and ordering said fragments based on said read numeric identifiers.

28. The method of claim 27, wherein at least one of:
a. said method further comprises removing said numeric identifiers from said fragments and concatenating said fragments in an order established by said read numeric identifiers;
b. said reading a numeric identifier in a plurality of fragments comprises decoding said numeric identifiers separately from said stored information; and
c. said reading comprises maximum likelihood decoding which ensures all ordinal numbers up to the total number of identifiers are represented in said numeric identifiers by selecting the numeric identifier with the highest probability of being each ordinal

29. (canceled)

30. (Cancelled)

31. The method of claim 24, wherein said correcting comprises employing an error correcting code decoder, optionally wherein said error correcting code decoder is a RS decoder.

32. (canceled)

33. A system comprising:
at least one hardware processor; and
a non-transitory computer-readable storage medium having stored thereon program code, the program code executable by the at least one hardware processor to perform a method of any one of claim 1.

34. A computer program product comprising a non-transitory computer-readable storage medium having program code embodied therewith, the program code executable by at least one hardware processor to perform a method of claim 1.
